(19) United States
(12) Patent Application Publication
(10) Pub. No.: US 2010/0099696 A1
(43) Pub. Date: Apr. 22, 2010

(54) Tamper resistant oral dosage forms containing an embolizing agent

(76) Inventors: Anthony Edward Soscia, Atlanta, GA (US); Yingxu Peng, Paoli, PA (US); Yichun Sun, North Potomac, MD (US); James R. Johnson, Germantown, TN (US); Atul J. Shukla, Cordova, TN (US); Wen Qu, Memphis, TN (US)

Correspondence Address: HOWARD EISENBERG, ESQ., 1220 LIMBERLOST LANE, GLADWYNE, PA, 19035, US

(21) Appl. No.: 12/587,771

(22) Filed: Oct. 13, 2009

Related U.S. Application Data
(60) Provisional application No. 61/196,227, filed on Oct. 16, 2008.

(51) Int. Cl. A61K 47/32; A61K 47/26; A61K 31/485; A61P 25/36

(52) U.S. Cl. 514/282; 514/772.6; 514/772.3; 514/777

(57) ABSTRACT

Oral dosage form containing a therapeutically effective amount of a drug susceptible to abuse and an effective amount of an embolizing agent which causes the production of a solid or semi-solid embolus or blockage after tampering.

This application claims priority from pending U.S. provisional patent application Ser. No. 61/196,227, filed on Oct. 16, 2008.

### FIELD OF THE INVENTION

The invention pertains to the field of pharmaceutical compositions that contain systems to deter tampering and abuse of therapeutic agents.

### BACKGROUND

Therapeutic pharmaceuticals are often the subject of illicit tampering and abuse. Illicit use of pharmaceutical products occurs when an individual knowingly tampers with a dosage form and administers it for a use that is not indicated with its dosing instructions.

The purpose for the illicit tampering of an oral pharmaceutical dosage form may be to increase the bioavailability of the drug by injecting or insufflating if the drug has lower gastrointestinal absorption. For example, typical abuse of a an oral dosage form may include chewing, crushing, and grinding with or without the aid of a mechanical device such as a coffee grinder, hammer, household blender or similar devices, then either insufflating the resultant powder or mixing the resultant powder or the intact dosage form with a suitable household solvent such as water, alcohol or vinegar and then injecting the solution intravenously, subcutaneously or rectally.

The illicit use of controlled prescription pharmaceuticals is currently an area of increasing concern. Many classes of therapeutic drugs are considered to be tamper prone therapeutics. For example, dosage forms containing opioid therapeutic agents are often the source of the illicit tampering.

Opioids include a diverse group of drugs, natural and synthetic, that have opium- or morphine-like properties and that bind to one of several subspecies of opioid receptors in the body. These drugs produce their major effects on the central nervous system and bowel. The effects of opioids or pseudo-opioids are remarkably diverse and include analgesia, drowsiness, changes in mood, and alterations of the endocrine and autonomic nervous systems. Opioid analgesics comprise the major class of therapeutic agents utilized in the management of moderate to severe pain.

In some individuals, opioids alter mood and feeling in a manner so as to provide a desirable sense of euphoria, often referred to as a “high”, which is disconnected to the therapeutic ameliorative effects of the drug in the dosage form. This euphoria is found by some individuals to be psychologically and somatically desirable. In addition, after repeated administration, some users develop a craving for re-administration of the opioid. The intensity of this craving may range from a mild desire to use the drug to a preoccupation with its procurement and use, not for its therapeutic ameliorative effects, but rather for its mood-altering effects. In the latter case, the opioid becomes the central fixation in a state commonly referred to as “drug abuse,” a term used to describe the usage of any drug in a manner which deviates from approved medical or social patterns within a given society. When the drug abuse involves overwhelming involvement with the use of the drug, securing its supply, and a high tendency to relapse into drug use after its withdrawal, an “addiction” is said to have developed.

Palermo, U.S. Pat. No. 6,228,863; Sackler, U.S. Pat. No. 7,157,103; Gruber, U.S. Pat. No. 7,214,385; and Mehta, U.S. Patent Application Publication 2004/0202717 describe the abuse of opioid narcotics by physical and chemical tampering.

Several attempts have been proposed to curtail abuse and tampering of opioids by pharmacological methods. The main attempts have included: (1) inclusion of additives such as aversive agents along with the active pharmaceutical ingredients, as described by the above Palermo, Sackler, Gruber and Mehta patents and application, and (2) modification of the delivery carrier of the active pharmaceutical ingredient.

Pharmaceutically acceptable additives useful to deter tampering and illicit use employed in the prior art have included drug antagonists, emetic agents, dyes, irritants, gelling agents and bittering agents, referred to in the prior art collectively as aversive agents. Inclusion of aversive agents of the prior art has several shortcomings. Aversive agents that act on the peripheral senses such as taste smell or physical perception are easily bypassable by the illicit user. Aversive agents that work via internal or central nervous system senses or receptors, such as emetic agents and drug antagonists, are more effective and more difficult for the illicit user to bypass. However, these agents may be problematic for the legitimate user unless the aversive agent is formulated with the drug in such a way that the aversive agent is not released in a significant quantity unless the dosage form is tampered with, since, for example even a small amount of a bioavailable aversive agent such as an emetic agent, may cause nausea. Therefore a more ideal agent should not interfere with the efficacy of the drug in a legitimate patient when a dosage form containing the drug is taken as intended by the manufacturer.

Pachter, U.S. Pat. Nos. 3,773,955; and 3,966,940, describe formulations containing a combination of opioid agonists and antagonists, in which the antagonist does not block the therapeutic effect when the admixture is administered orally but prevents analgesia, euphoria or physical dependence when administered parenterally by an abuser. Gordon, U.S. Pat. No. 4,457,933 describes a method for decreasing both the oral and parenteral abuse potential of strong analgesic agents by combining an analgesic dose of the analgesic agent with an antagonist in specific, relatively narrow ratios. Kaiko, U.S. Pat. Nos. 6,277,384; 6,375,957; and 6,475,494 describes oral dosage forms including a combination of an orally active opioid agonist and an orally active opioid antagonist in a ratio that, when delivered orally, is analgesically effective but that is aversive in a physically dependent subject. While such a formulation may be successful in deterring abuse, it also has the potential to produce adverse effects in legitimate patients due to leaching of antagonist from the dosage form when taken as intended. Sackler, U.S. Pat. No. 7,157,103, discloses the use of a bittering agent, irritant and/or gelling agent which releases when a dosage form is tampered with.

Shaw, U.S. Pat. No. 3,980,766; Hoffmeister, U.S. Pat. No. 4,070,494; and Bastin, U.S. Pat. No. 6,309,668, describe the approach of modification of the delivery carrier of the active pharmaceutical ingredient. The formulations in these patents were designed to prevent the injection of compositions meant for oral administration through the use of water gelable agents. Shaw describes the incorporation of an ingestible solid which causes a rapid increase in viscosity upon exposure to an aqueous solution thereof. Hoffmeister describes the incorporation of a non-toxic, water gelable material in an amount sufficient to render the drug resistant to aqueous extraction. Bastin describes a tablet for oral administration containing two or more layers comprising one or more drugs and one or more gelling agents within each separate layer of the tablet. The resulting tablet forms a gel when combined with the volume of water necessary to dissolve the drug. This formulation thus reduces the in vitro extractability of the drug from the tablet.

Launchbury, European Patent EP0502042 describes a soft gelatin capsule shell or a two-piece hard gelatin capsule and a fill comprising a benzodiazepine drug dissolved or suspended in a gel, wherein the gel has a drop point (° C.), prior to encapsulation, of at least 46 and comprises at least 63% by weight of polyethylene glycol 600, at least 2% by weight of polyethylene glycol 4000 or 6000 and at least 21% by weight of an intermediate polyethylene glycol having an average molecular weight between 600 and 4000, the gel consisting of polyethylene glycol. This formulation is meant to decrease the syringeability of the drug.

The aforementioned addition of gelling agents may form a viscous or gel like mass when an aqueous solution is added or may decrease syringeability, thereby making the act of injection of a gelled material difficult. However, such methods fail to address the potential for abuse by the addition of a solvent suitable for injection that does not gel the agent and in which the active ingredient or agent may be soluble or partially soluble or suspendable, thus allowing potential separation of the gelling agent and the drug or injection of the drug either intravenously, subcutaneously or intramuscularly.

Embolizing agents and their use have been disclosed in the prior art. Examples of suitable embolizing agents are discussed in Interventional Radiology By R. F. Dondelinger, Olga B. Adler, P. Rossi Contributor R. F. Dondelinger, Olga B. Adler Published by Thieme, 1990ISBN 086577286X, 9780865772861, and is incorporated herein in its entirety. As described in the prior art, embolizing agents are designed to form an embolism or blockage upon application to non-enteral bodily fluids, usually the blood, under certain body conditions such as temperature, biological signals or pH. Preferably the embolus is formed at the site of administration so as not to cause harm to a patient. Typically, the embolizing agent is introduced into the blood vessel of a patient so as to prevent bleeding from the open end of the blood vessel or to facilitate cure of a disordered blood vessel without undertaking a surgical procedure with embolization of the disordered blood vessel by the subsequent coagulation or solidification of the embolizing agent.

As described in the prior art, an embolizing agent in either a solid or liquid form is combined with an embolizing liquid in which the embolizing agent is soluble or suspendable in or is heated above the temperature of the body cavity where the embolizing agent is to be injected. The solubilized embolizing agent is then injected into the area of the body or vascular system where the practitioner desires a block or occlusion.

Once in the body cavity or vascular position, the embolizing agent may solidify or coagulate by several mechanisms. The commonly described mechanisms are; 1) the embolizing fluid will diffuse into the surrounding tissue or into the vascular system thereby precipitating the embolizing agent, 2) the embolizing agent, which is temperature sensitive, will cool and solidify upon contact with the body cavity or blood supply, 3) the solution containing the embolizing agent is at a higher or lower pH than the body cavity or vasculature and the embolizing agent is designed to solidify when it is exposed to the pH of the body cavity or the vascular system, 4) biological signaling may occur in the case of an agent that signals platelet adhesion or another occluding factor such as in the case with haemostatic agents.

Greff, U.S. Pat. No. 5,667,767 discloses compositions suitable for use in embolizing blood vessels which comprise an ethylene vinyl alcohol copolymer, a biocompatible solvent and a water insoluble contrasting agent selected from the group consisting of tantalum, tantalum oxide and barium sulfate. Greff, U.S. Pat. No. 5,580,568 discloses compositions suitable for use in embolizing blood vessels which comprise a cellulose diacetate polymer, a biocompatible solvent and a water insoluble contrasting agent selected from the group consisting of tantalum, tantalum oxide and barium sulfate. Ji, U.S. Pat. No. 5,894,022 describes an injectable embolic material for occlusion of vascular elements and fallopian tubes produced by preparing an aqueous matrix base comprising an aqueous solution of a matrix material selected from the group consisting of albumin, gelatin, fibrinogen, lactoglobulin, immunoglobulin, actin, and acrylamide. Kunihiro, U.S. Pat. No. 5,149,540 describes a stabilized thrombin which can be utilized for oral purposes. Evans, U.S. Pat. No. 6,342,202 discloses compositions suitable for use in embolizing blood vessels which compositions comprise a polymer, a biocompatible solvent and a contrast agent. The polymer is selected from the group consisting of polyacrylonitrile, polyurethane, polyvinylacetate, cellulose acetate butyrate, nitrocellulose and copolymers of urethane/carbonate and copolymers of styrene/maleic acid. Klein, U.S. Pat. No. 6,355,275 Describes methods of embolization using an embolizing agent composition that includes microparticles with carbon surfaces, and comprising a contrast agent. Preferred microparticles include a permanently radiopaque particle substrate and a pyrolytic carbon surface. Haemostic materials may be preferably employed as the embolizing agents. Hardy, International Application No. PCT/GB2007/004382 describes the use of haemostatic agents which may be employed in the present invention. One method of manufacture of the chitosan-based haemostatic material involves preparation of an active base material by preparing a mixture of chitosan and acid in a solvent in which the chitosan is insoluble (usually 80:20 ethanol:water). Where used, a surfactant may also be added to this mixture. The solvent is evaporated to provide a substantially dry active base material. The active base material may then optionally be combined with other materials such as inert materials to provide the haemostatic powder. Thus, the chitosan-based haemostat may comprise a powder of a chitosan salt, optionally in combination with an inert material and/or a medical surfactant. An example of a suitable commercially available chitosan-based haemostat is Celox™ (MedTrade Products Limited).

Temperature and pH sensitive polymers have also been suggested as embolizing agents. Ito, U.S. Pat. No. 5,525,334 describes the use of a thermosensitive polymer, which is liquid at low temperatures but causes coagulation when heated up to the body temperature of the patient, into the blood vessel followed by in situ heating of the solution. The thermosensitive polymer found to be suitable for the purpose is a homopolymer or copolymer of an N-substituted (meth) acrylamide monomer having a specified intrinsic viscosity in tetrahydrofuran and gives an aqueous solution capable of exhibiting phase transition from a liquid to a coagulate at a transition temperature of 10° to 37° Gutowska, U.S. Pat. No. 6,979,464 describes a therapeutic agent carrier having a thermally reversible gel or gelling copolymer that is a linear random copolymer of an [meth-]acrylamide derivative and a hydrophilic comonomer, wherein the linear random copolymer is in the form of a plurality of linear chains having a plurality of molecular weights greater than or equal to a minimum gelling molecular weight cutoff and a therapeutic agent. Yamashita K, Taki W, Iwata H, et al. A cationic polymer, Eudragit-E, as a new liquid embolic material for arteriovenous malformations. Neuroradiology 1996; 38(suppl 1):S151-56 describes the use a pH sensitive copolymer of methyl and butyl methacrylate and dimethylaminoethyl methacrylate (Amino Methacrylate Copolymer). Amino Methacrylate Copolymer is a cationic copolymer that is supplied as off-white granules. This copolymer consists of butyl methacrylate, dimethylaminoethyl methacrylate and methyl methacrylate in the ratio of 1:2:1.

Therefore, a significant need remains for additional ways to provide a dosage form that reduces or eliminates the potential for abuse of a drug by tampering with the dosage form containing the drug and then administering the tampered dosage unit by a route other than that which it is intended by the manufacturer.

### DESCRIPTION OF THE INVENTION

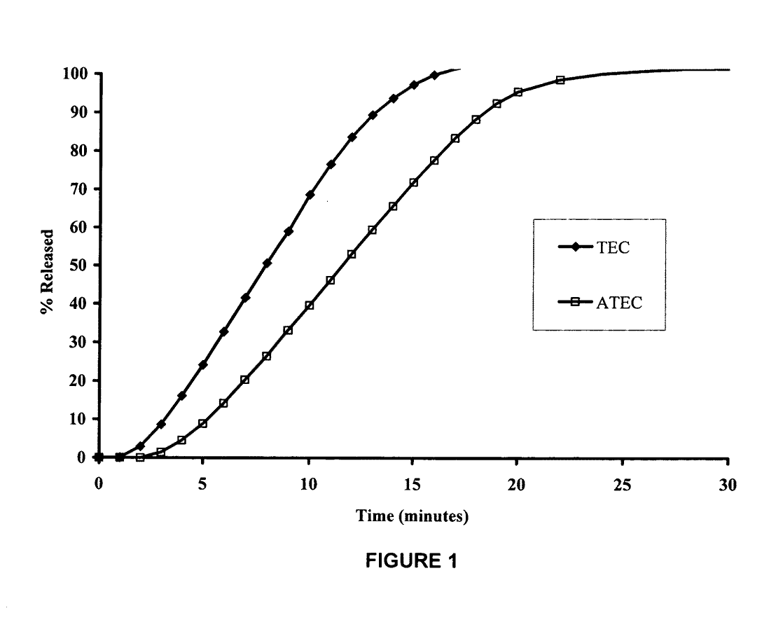

FIG. 1 is a graph showing the effect of varying hydrophilicity/hydrophobicity of plasticizers on the release of Oxycodone HCl from gels in SGF (simulated gastric fluid). The composition of the gel is drug=20%, Eudragit E:ATEC=40:60 w/w.

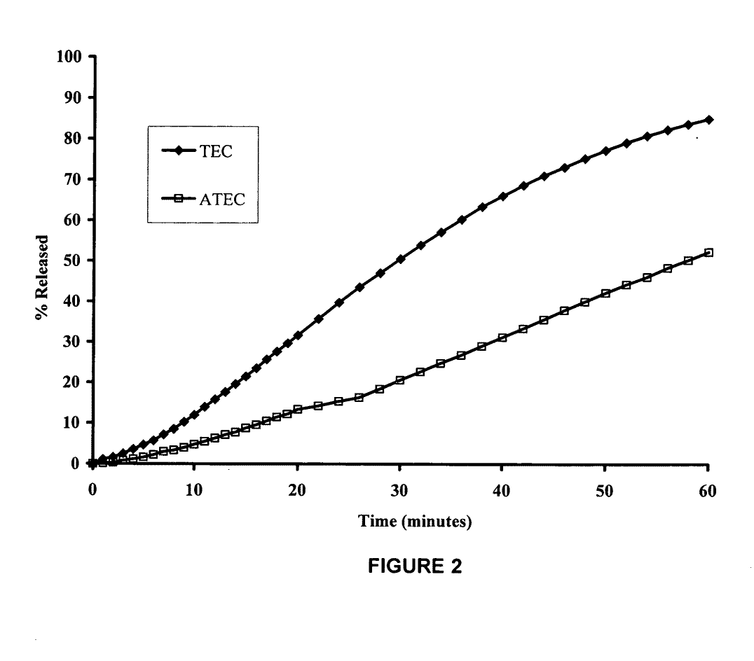

FIG. 2 is a graph showing the effect of varying hydrophilicity/hydrophobicity of plasticizers on the release of Oxycodone HCl from gels in water. The composition of the gel is drug=20%, Eudragit E:ATEC=40:60 w/w.

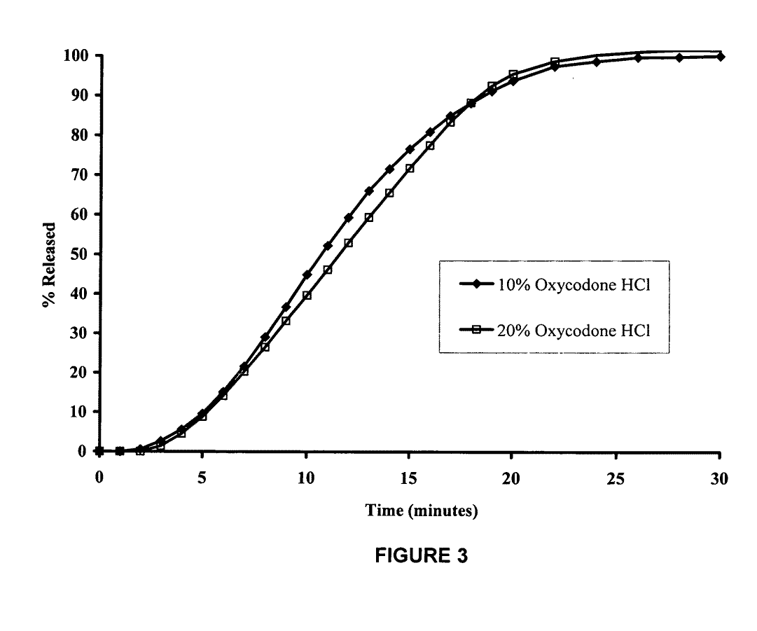

FIG. 3 is a graph showing the effect of varying loading level of drug on the release of Oxycodone HCl from gels in SGF. The composition of the blank gel is Eudragit E:ATEC=40:60 w/w.

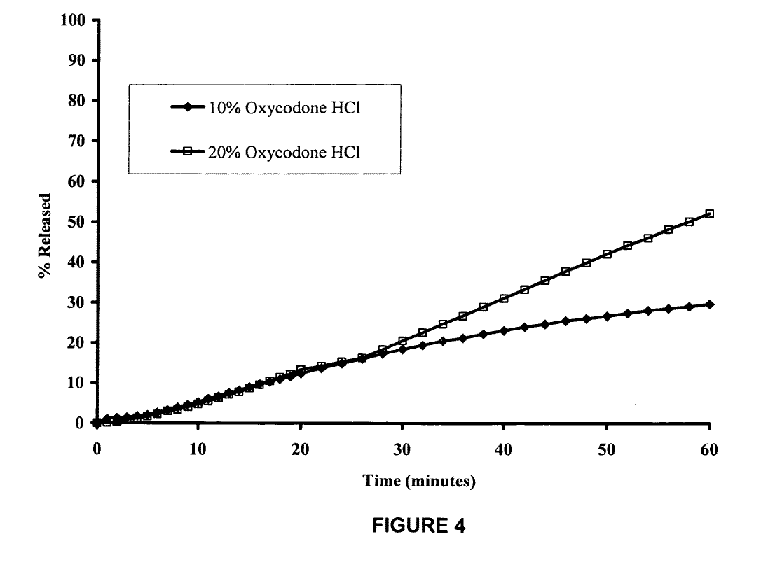

FIG. 4 is a graph showing the effect of varying loading level of drug on the release of Oxycodone HCl from gels in water. The composition of the blank gel is Eudragit E:ATEC=40:60 w/w.

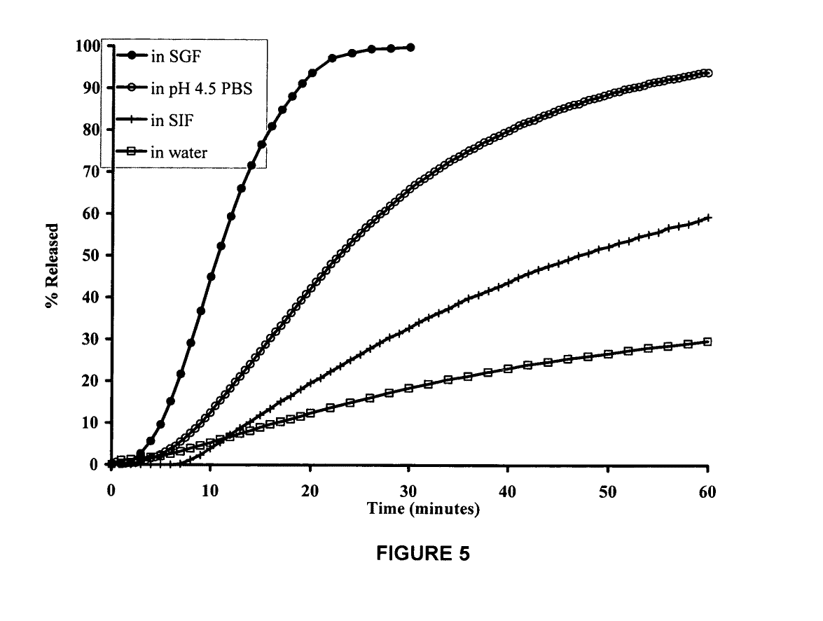

FIG. 5 is a graph showing the effect of dissolution medium on the release of Oxycodone HCl from gels. The composition of the gel is Oxycodone HCl=10%, Eudragit E:ATEC=40:60 w/w.

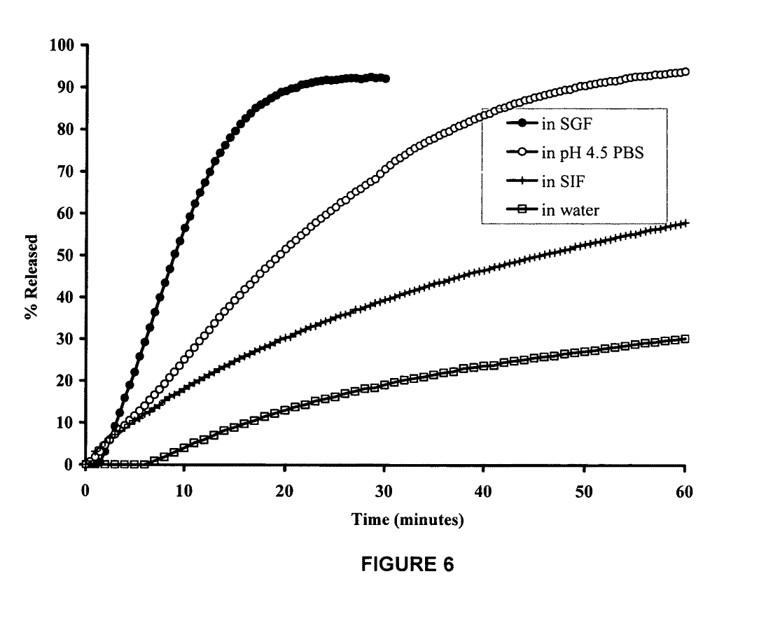

FIG. 6 is a graph showing the release of drug from tampered Oxycodone HCl-loaded gels in different medium. The composition of the gel is Oxycodone HCl=10%, Eudragit E:ATEC=40:60 w/w.

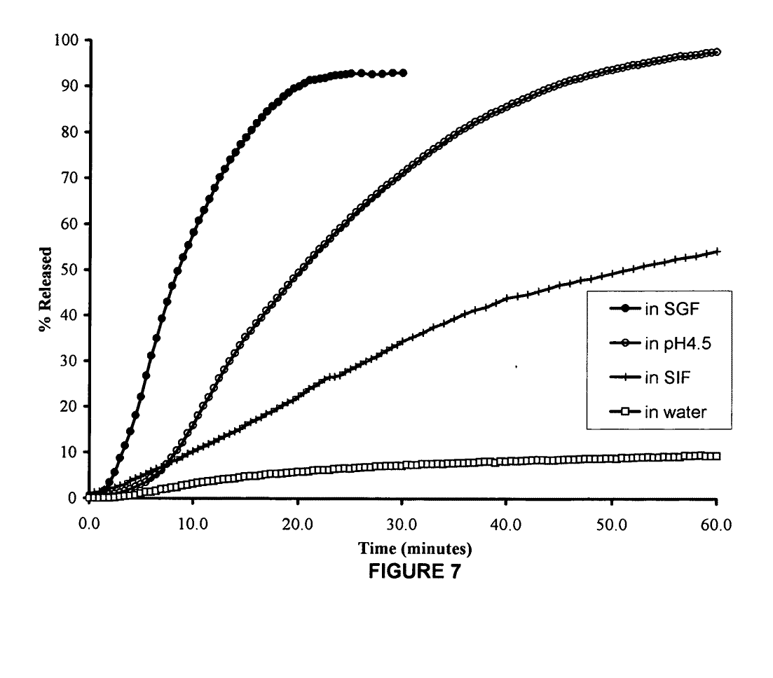

FIG. 7 is a graph showing the release of drug from Oxycodone base-loaded gels in different medium (oxycodone base=10%, Eudragit E:ATEC=40:60 w/w).

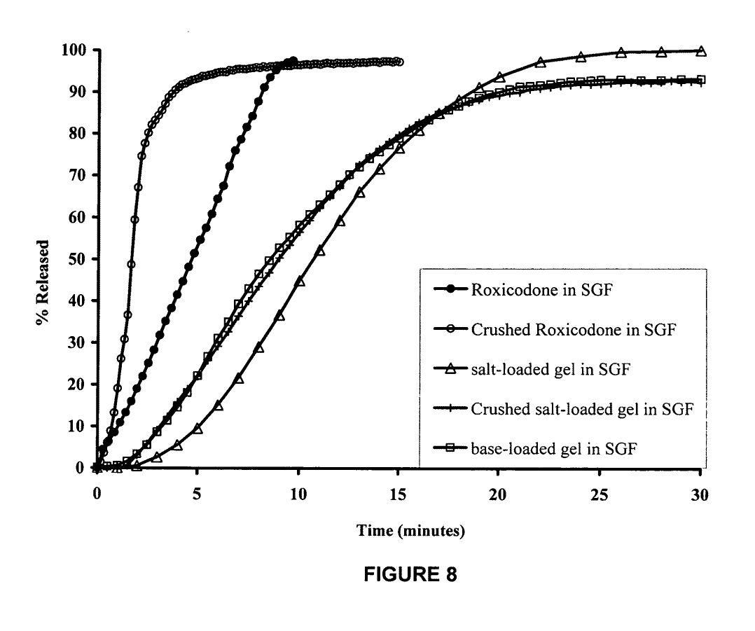

FIG. 8 is a graph showing the release of Oxycodone from different formulations in SGF. The composition of the gel is drug=10%, Eudragit E:ATEC=40:60 w/w.

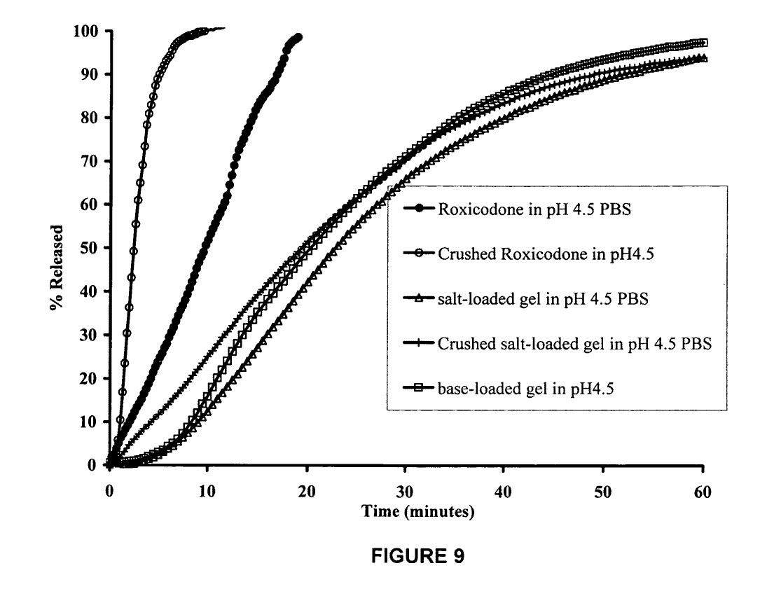

FIG. 9 is a graph showing the release of Oxycodone from different formulations in pH4.5 PBS. The composition of the gel is drug=10%, Eudragit E:ATEC=40:60 w/w.

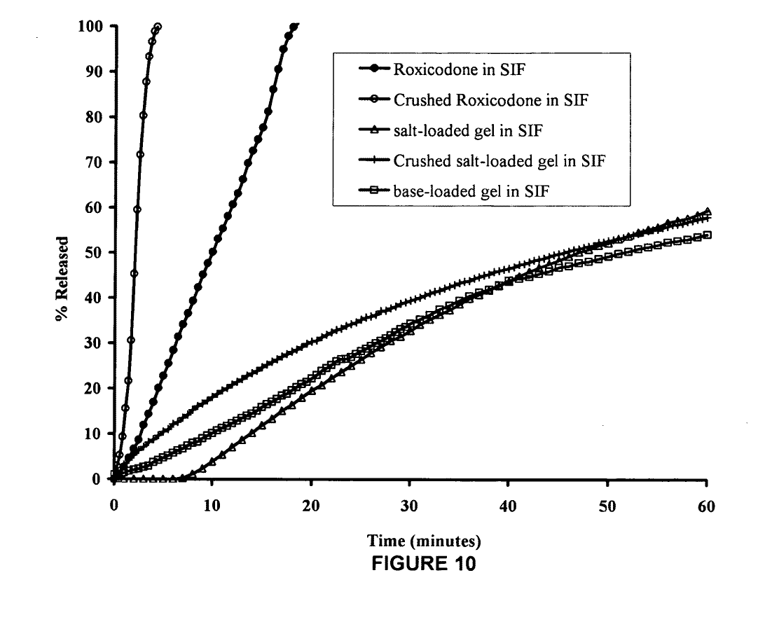

FIG. 10 is a graph showing the release of Oxycodone from different formulations in SIF. The composition of the gel is drug=10%, Eudragit E:ATEC=40:60 w/w.

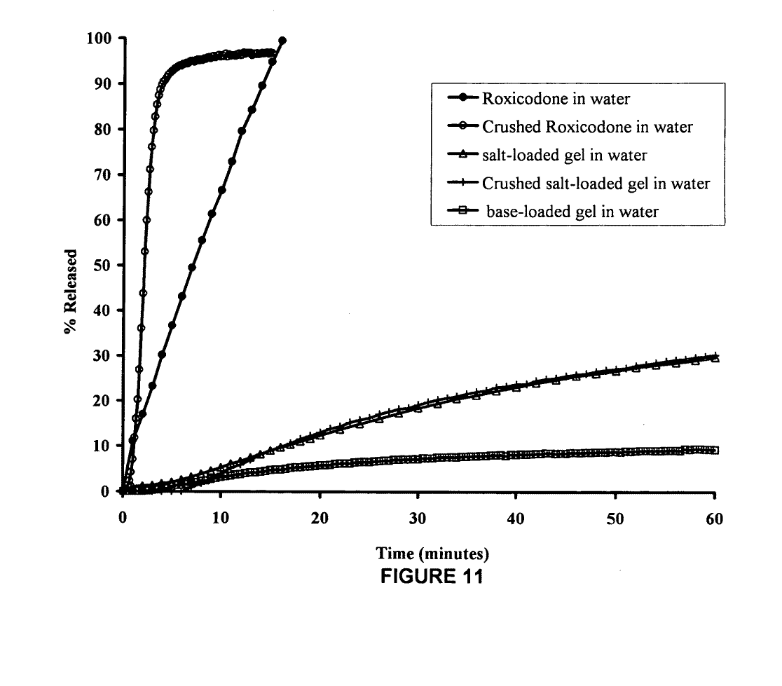

FIG. 11 is a graph showing the release of Oxycodone from different formulations in water. The composition of the gel is drug=10%, Eudragit E:ATEC=40:60 w/w.

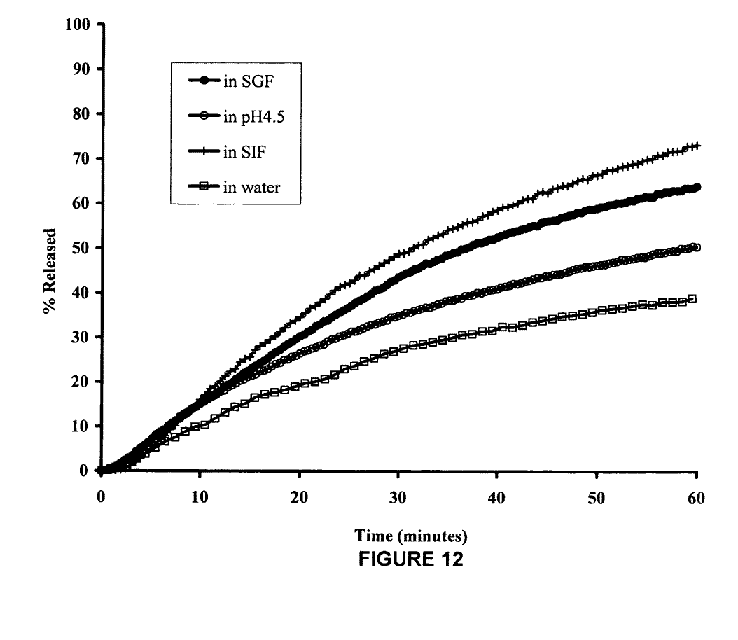

FIG. 12 is a graph showing the release of Oxycodone from gels prepared with Eudragit L100. The composition of the gel is Oxycodone HCl=10%, Eudragit L100:ATEC:Tetraglycol=1:3:3 w/w.

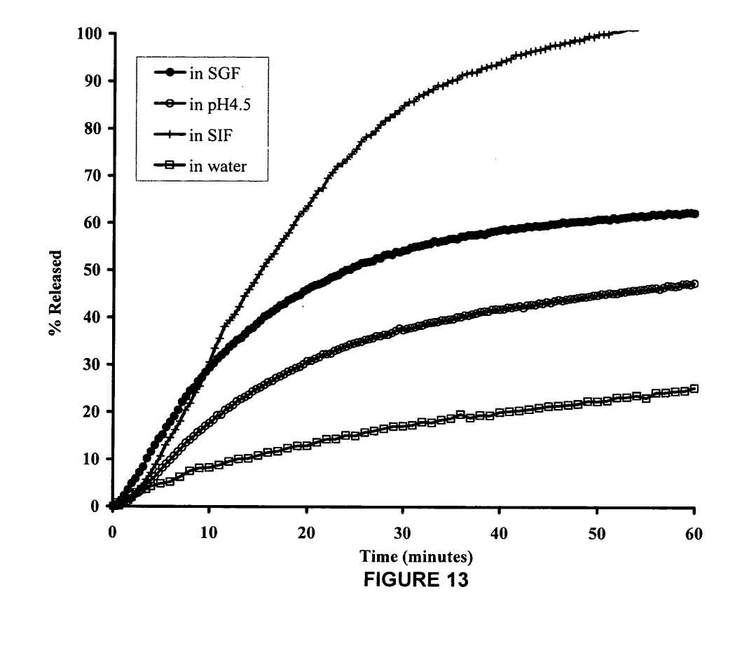

FIG. 13 is a graph showing the release of Oxycodone from gels prepared with Eudragit L100-55. The composition of the gel is Oxycodone HCl=10%, Eudragit L100-55:ATEC:Tetraglycol=1:3:3 w/w.

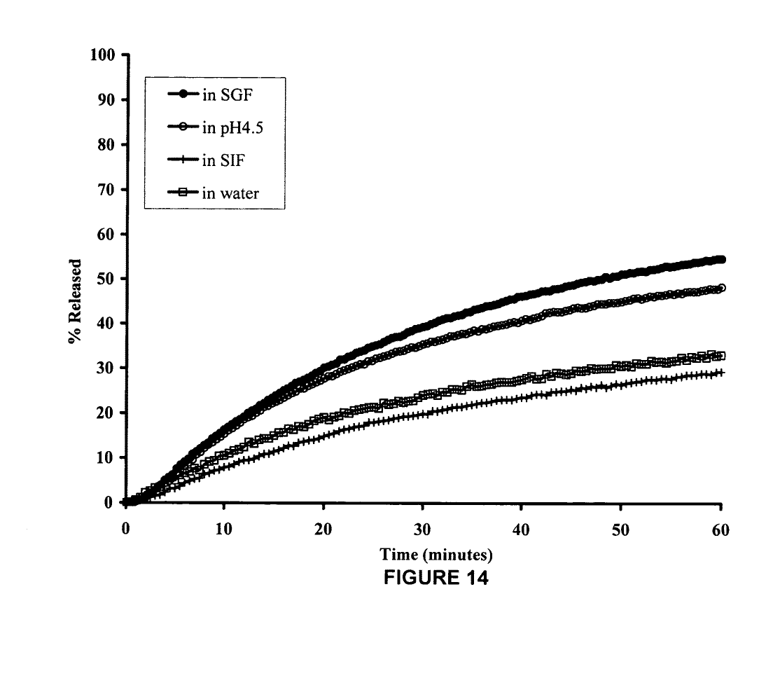

FIG. 14 is a graph showing the release of Oxycodone from gels prepared with Eudragit 5100. The composition of the gel is Oxycodone HCl=10%, Eudragit S100:ATEC:Tetraglycol=1:3:3 w/w.

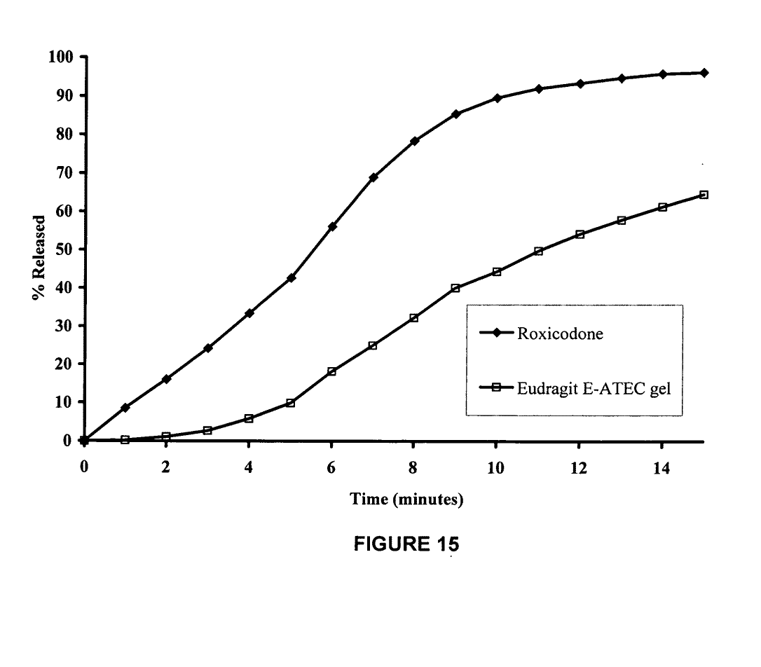

FIG. 15 is a graph showing the release of Oxycodone HCl from different formulations in 40% ethanol. The composition of the gel is Oxycodone HCl %=10%, Eudragit E:ATEC=40:60 w/w).

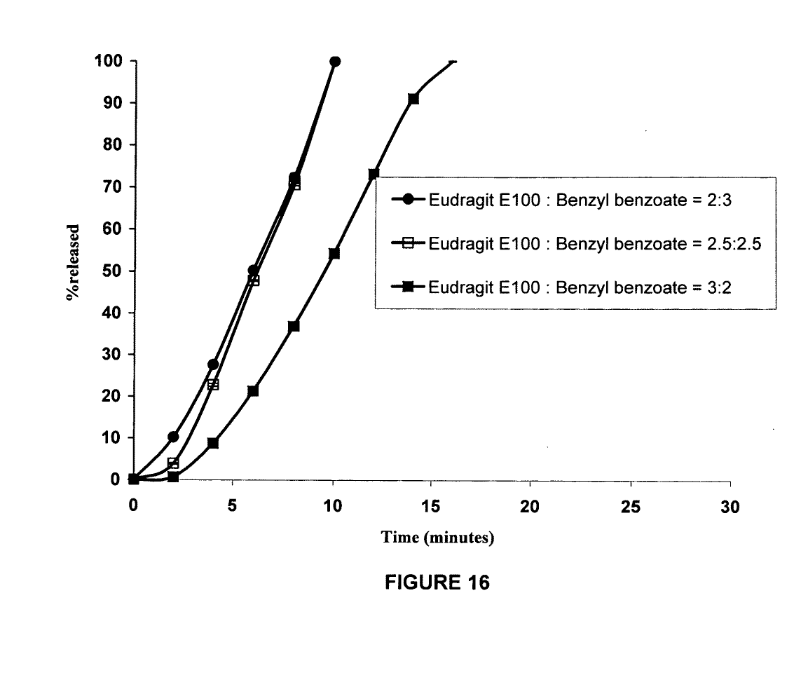

FIG. 16 is a graph showing the effect of the ratio of Eudragit E100:solvent on the release of oxycodone HCl from gels in SGF. (Oxycodone HCl=10%).

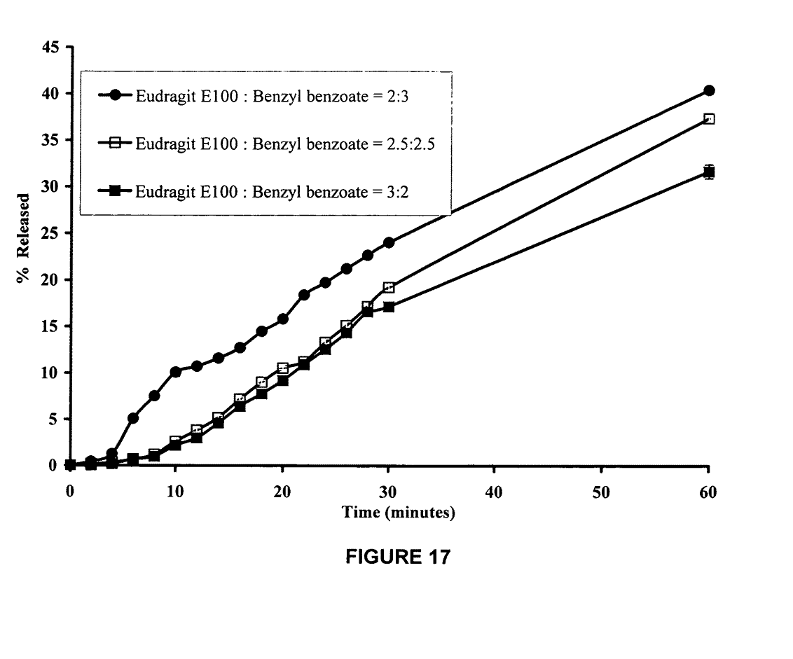

FIG. 17 is a graph showing the effect of the ratio of Eudragit E100:solvent on the release of oxycodone HCl from gel in water. (Oxycodone HCl=10%).

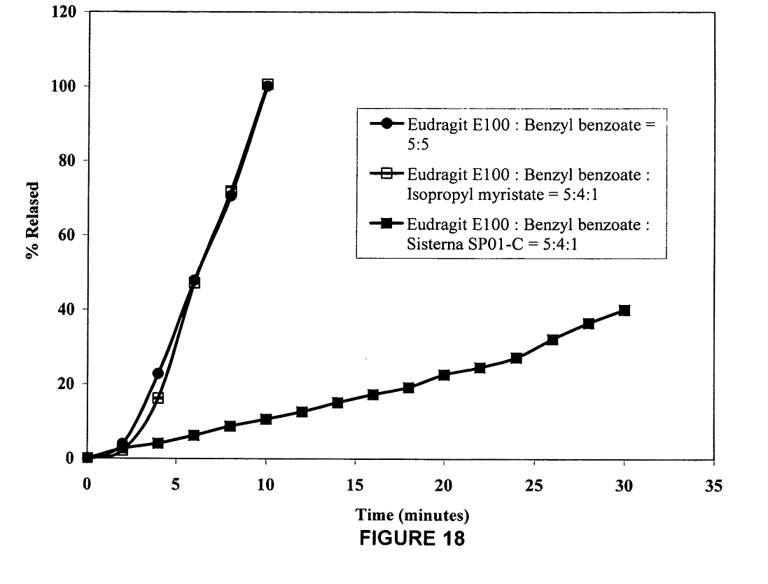

FIG. 18 is a graph showing the effect of solvent blends and hydrophobic additives on the release of oxycodone HCl from gels in SGF. (Oxycodone HCl=10%).

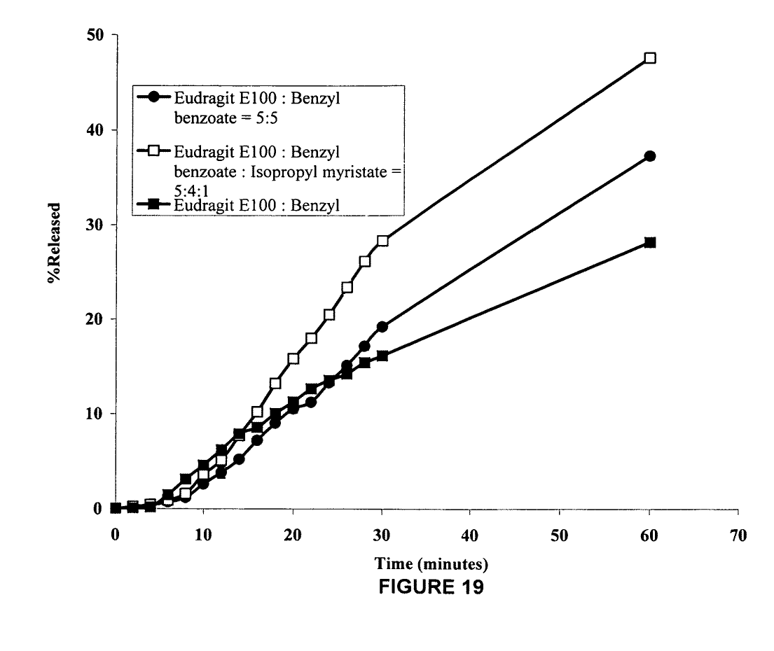

FIG. 19 is a graph showing the effect of solvent blends and hydrophobic additives on the release of oxycodone HCl from gels in water. (Oxycodone HCl=10%).

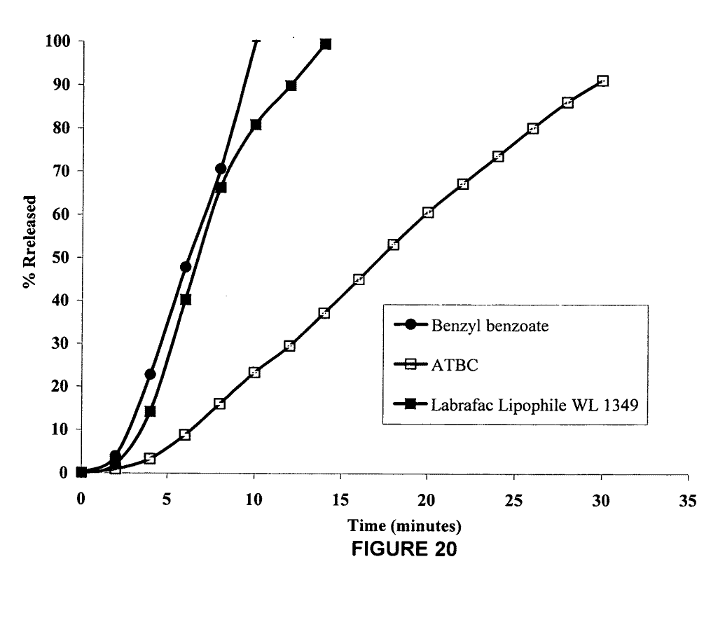

FIG. 20 is a graph showing the Effect of hydrophobicity of plasticizer on the release of oxycodone HCl from gels in SGF. The composition of the gel is drug=10%, Eudragit E100:plasticizer=45:45 w/w.

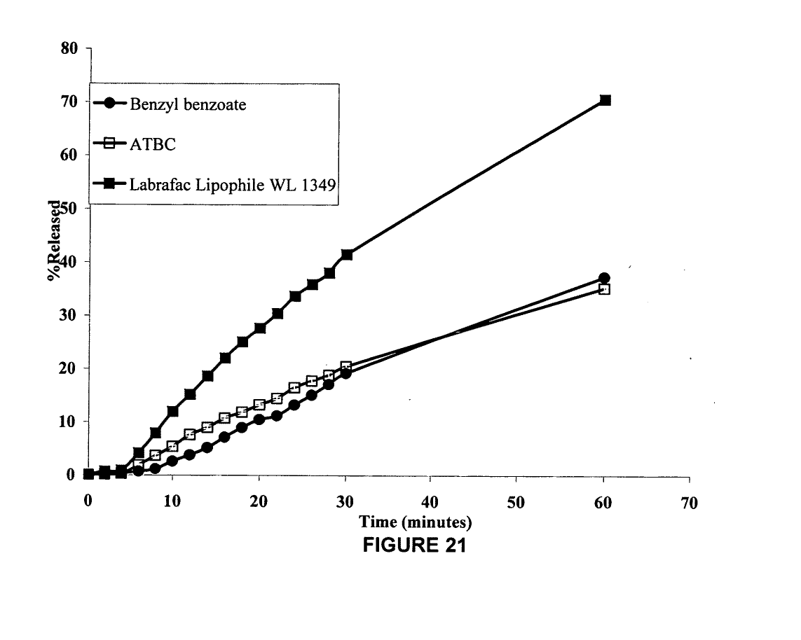

FIG. 21 is a graph showing the effect of hydrophobicity of plasticizer on the release of oxycodone HCl from gels in water. The composition of the gel is drug=10%, Eudragit E100:plasticizer=45:45 w/w.

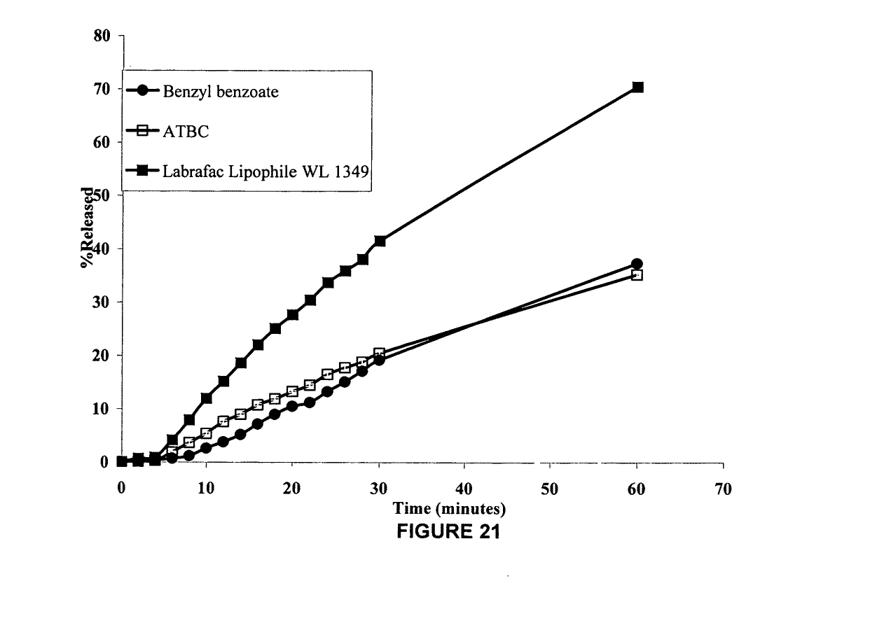

FIG. 22 is a graph showing the release in SGF of oxycodone base from different formulations containing different sustained releasing agents, in this case carbomer gelling agents having differing viscosities, and containing one or more plasticizers of varying degrees of hydrophilicity/hydrophobicity.

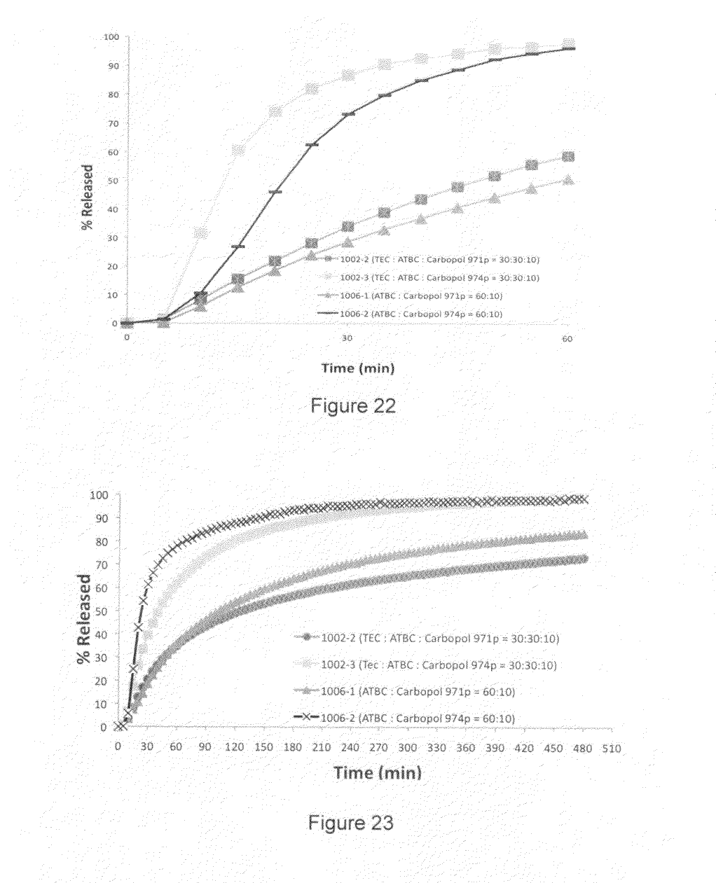

FIG. 23 is a graph showing the release in pH 4.5 buffer of oxycodone base from different formulations containing different sustained releasing agents, in this case carbomer gelling agents having differing viscosities, and containing one or more plasticizers of varying degrees of hydrophilicity/hydrophobicity.

### DESCRIPTION OF THE INVENTION

It has been discovered that the inclusion of an embolizing agent as part of a dosage form containing a drug is effective in forming an embolus or blockage in fluids characteristic of non-gastrointestinal (non-GI) fluids, such as fluids of the nasal cavity, interstitial fluid or blood after said dosage form has been tampered with and applied to the non-GI site of administration. The formation of said embolus reduces, diminishes or eliminates the “attractiveness” of further or continued tampering by forming a solid or semi-solid mass in non-GI fluids.

The formation of the embolus at non-GI fluids has one or more effects that deter further or continuing abuse of the drug. For example, the embolus may reduce or retard release of the drug from the tampered dosage form due to re-solidification of the embolizing agent which in turn may slow diffusion of the drug into the body. For another example, the embolus may occlude or block the route of administration, such as the nasal passages or vein into which the tampered dosage form is administered. Thus, the formation of the embolus in accordance with the invention reduces or eliminates the sensation of euphoria that would otherwise be obtained by an individual who illicitly tampers with the dosage form in order to abuse the drug.

The embolizing agent or agents and the drug, may be in physical association as part of the dosage form or may exist as separate and distinct elements of the dosage form.

The term “tampered dosage form” and “tampered”, when referring to an oral dosage form, are used interchangeably herein and are defined for purposes of the present invention to mean that the oral dosage form has been manipulated by mechanical, thermal, and/or chemical means which changes the physical properties of the dosage form, e.g., to liberate the active ingredient and allow for injection or insufflation, or to make the active ingredient available for inappropriate use, such as administration by an alternate route, e.g., rectally. The tampering can be, e.g., by means of crushing, shearing, grinding, chewing, and/or dissolution in a solvent suitable for injection that the embolizing agent is soluble or suspendable in, heating, (e.g., greater than about 45° C.), or any combination thereof.

The terms “substantially non-releasable form” and “sequestered” for purposes of the present invention when referring to an aversive agent or an embolizing agent, mean that the aversive agent or embolizing agent is not released or is substantially not released at one hour after the intact dosage form containing an opioid agonist and at least one embolizing agent is orally administered (i.e., without having been tampered with).

The terms “not released” or “substantially not released” when referring to an aversive agent or embolizing agent indicate that an individual who orally administers an intact dosage form containing the aversive agent or embolizing agent will not be adversely affected by the aversive agent or embolizing agent. In accordance with the present invention, an embolizing agent in a substantially non-releasable form may be prepared in accordance with the teachings of Sackler in U.S. Pat. No. 7,157,103, the disclosure of which is hereby incorporated by reference in its entirety, which describes a dosage form comprising an aversive agent such as a irritant in a substantially non-releasable form. For purposes of the present invention, the amount released after oral administration of an intact dosage form may be measured in-vitro via the dissolution at 1 hour of the dosage form in 900 ml of Simulated Gastric Fluid using a USP Type II (paddle) apparatus at 75 rpm at 37° C. Such a dosage form is also referred to as comprising a “sequestered embolizing agent” depending on the agent or agents which are not released or substantially not released. In certain preferred embodiments of the invention, the substantially non-releasable form of the embolizing agent is resistant to laxatives (e.g., mineral oil) used to manage delayed colonic transit and resistant to achlorhydric states. In a preferred embodiment, the embolizing agent is not released or not substantially released 4, 8, 12 and/or 24 hours after oral administration.

The term “analgesic effectiveness” is defined for purposes of the present invention as a satisfactory reduction in or elimination of pain, along with a tolerable level of side effects, as determined by the human patient.

The term “sustained release” is defined for purposes of the present invention as the release of a drug, such as an opioid analgesic, from an oral dosage form at such a rate that blood (e.g., plasma) concentrations (levels) are maintained within the therapeutic range but below toxic levels over an extended period of time, e.g., from about 12 to about 24 hours as compared to an immediate release product. Preferably the sustained release is sufficient to provide a twice-a-day or a once-a-day formulation.

The term “particles” refers to granules, spheroids, beads or pellets. In certain preferred embodiments, the particles are about 0.2 to about 2 mm in diameter, more preferably about 0.5 to about 2 mm in diameter.

The term “parenterally” as used herein means administration by other than swallowing followed by absorption in the stomach or intestines, and includes subcutaneous injections, intravenous injections, intramuscular injections, intrasternal injections, infusion techniques, or other methods of injection known in the art.

The terms “insufflated,” “inhaled,” and “snorted,” are used interchangeably herein and include trans-mucosal, trans-bronchial, and trans-nasal administration.

The terms “embolizing agent” and “coagulating agent” are used interchangeably herein to mean an agent that is, or that includes, a compound or composition that imparts a semi-solid or solid quality to a tampered dosage form upon the addition of fluid in which the compound or composition is soluble or suspendable and the exposure of the compound or composition to a non-oral area of the body where the solubilized compound or composition will precipitate, solidify or coagulate due to one or more of the following factors: diffusion of the fluid into the surrounding tissues or bodily fluids; a change in pH between the non-oral area of the body and the compound or composition; a temperature differential between the non-oral area of the body in which the drug is administered and the compound or composition; or a biological condition which induces coagulation or solidification, such as platelet aggregation.

The term “embolizing liquid” means any liquid solvent that the embolizing agent is fully or partially soluble or suspendable in. The liquid solvent typically is a solvent that an illicit user would find suitable for injection or insufflation.

The term “unsuitable for injection” is defined for purposes of the present invention to mean that one would have substantial difficulty injecting or insufflating the dosage form (e.g., due to pain upon administration, sudden precipitation of the embolizing agent in the nasal cavity or difficulty pushing the liquefied dosage form through a blocked vein due to the sudden precipitation, coagulation and blockage imparted by the embolizing agent, thereby reducing the potential for abuse of the opioid analgesic in the dosage form).

As used herein, the term “does not significantly impede release of a drug from a dosage form” in relation to an embolizing agent means that the rate of release of the drug from the dosage form with an embolizing agent is similar to that from an otherwise identical dosage form without an embolizing agent.

As used herein, the term “burst release” is synonymous with the term “dose dumping” and means the increased percent or amount release of a drug from a tampered dosage form within a given time interval compared with that from a dosage form that is administered in an untampered form and in a manner intended by the manufacturer of the dosage form. The percent or amount of a drug released per unit time is also referred to as “rate of release” of the drug. Burst release typically occurs from dosage forms that have been tampered with, especially from extended-release dosage forms that have been tampered with. The duration of the time of the burst may be any time interval, such as from 1 second to 2 minutes, from 2 minutes to 20 minutes, or from 20 minutes to 60 minutes or more, from the initiation of release of drug from the dosage form.

The terms “drug,” “pharmaceutical agent,” “pharmaceutical,” “active pharmaceutical ingredient,” “active ingredient,” and “API” are used interchangeably herein and are synonymous with biologically active substances, which may be subject to tampering and abuse via injection or insufflation.

The dosage form of the invention is an oral dosage form, such as a tablet or capsule, containing a drug and an embolizing agent which is present within the dosage form in an amount sufficient to produce an embolus or coagulation at the site of administration when the dosage form is tampered with and is subsequently administered to an individual.

In certain embodiments, the oral dosage forms of the present invention comprising a drug subject to being abused and an embolizing agent or agents as a component(s) of the dosage form helps to prevent or retard injection and/or insufflation of the drug within the dosage form, by decreasing the “attractiveness” of the dosage form to a potential abuser. In certain embodiments, the oral dosage forms of the present invention comprise a drug subject to tampering, a pH dependent embolizing agent and a pharmaceutically acceptable solvent. The dosage form, which may be an oral capsule, helps to prevent or retard injection and/or insufflation, by decreasing the “attractiveness” of the dosage form to a potential abuser.

In certain embodiments, the present invention is an embolizing agent which is not soluble in certain sections of the gastrointestinal tract but is soluble in alcohol, acidic or aqueous alcoholic solutions, or acidic solutions. Preferably, the embolizing agent will not interfere with the intended release of the drug in the dosage form when the dosage form containing the embolizing agent is taken orally and in its intact form, but will reduce or diminish the ability of the potential abuser to obtain a “high” when the dosage form is tampered with and subsequently injected or insufflated.

It has also been discovered that formulations prepared with pH sensitive polymers (with pH dependent water solubility), pharmaceutical solvents or plasticizers with or without additives have the function of embolizing agent when the dosage form is tampered with and injected and or insufflated. The aforementioned compositions can be prepared by mixing the required ingredients with or without the aid of volatile solvents. Volatile solvents such as acetone, ethyl acetate, etc. are removed using an appropriate process to prepare the finished drug products. The mixture can be processed mechanically with or without heat. The physical states of the formulations range from liquid to solid based on the composition of the formulations. The physical states of the formulation can be modulated by the ratio between the polymer and the solvent, molecular weight of the polymer, types of polymers or solvents, additives, etc. used in the formulations. The finished dosage form can be a solution, suspension, gel, film, capsules, beads, tablets, etc.

The active ingredient incorporated in such formulations can be released for intended use (immediate release or delayed-release or modified release) in the GI tract because of the unique pH environments in the GI tract. The pH of the stomach is approximately 1 to 3. The pH of the small intestine ranges from 5 to 7. Some of the polymers such as Eudragit E used in the formulations can only be dissolved in low pHs (the low pH present in stomach). Some of the polymers such as Eudragit L 100 and S 100 used in the formulations can be dissolved in relatively high pHs (higher than pH 5.5 in small intestine). If a single polymer (with pH dependent water solubility) such as Eudragit E or Eudragit L or Eudragit S is used to prepare the formulation, immediate release (if Eudragit E is used) or delayed-release (if Eudragit L or Eudragit S is used) of the loaded API can be obtained. If a combination of two or more polymers (with different pH dependent solubility such as Eudragit E and Eudragit S or with pH independent solubility such as ethylcellulose), a sustained release of the loaded API can be obtained.

Additives such as other polymers, wax, fillers (avicel, starch, cabosil, lactose, etc.), antioxidants, chelating agents, etc. may be added into the formulations to modulate the release of the API and/or enhance the stability, processibility, performance of the formulations or act as embolizing agent(s). The polymers (with pH dependent water solubility) or additives may precipitate after injection if the formulations are tempered with and then injected because of solvent change or pH change. In some cases, the additives present in the formulations can not be dissolved by the tempering solvents and/or body fluid, the small particles of the additives can work as embolizing agent.

The form of the APIs loaded into the formulations can be in non-ionized form, such as free acids or free base; or ionized forms such as salts. The API can also be complexed with other materials before being loaded into the formulations. Such additives include but are not limited to pharmaceutically acceptable antioxidant(s), such as vitamins E, butylated Hydroxytoluene, and monothioglycerol; chelating agent(s) such as EDTA; fillers such as microcrystalline cellulose and lactose; wax such as Carnauba wax, Microcrystalline wax, and white wax; polymer(s) such as ethylcellulose, Ethylene Vinyl Acetate, or Polyethylene Glycol; antitacking agent(s) such as talc and Colloidal Silicon Dioxide.

In certain embodiments, the dosage form of the present invention comprises an embolizing agent, such as a pH sensitive embolizing agent, which is soluble in certain pH ranges of the gastrointestinal tract but not soluble in the pH range of the blood or of the nasal cavity. In a preferred embodiment, the pH sensitive embolizing agent is soluble in the pH of the intended compartment of the gastro-intestinal tract and does not significantly impede release of a drug from a dosage form when the dosage form is taken as intended by the manufacturer but is precipitated or coagulated when the dosage form is administered in a tampered form and provides a solid or semi-solid quality to the tampered dosage form which slows, prevents or delays the diffusion or absorption of the opioid analgesic from the dosage form after injection or inhalation such that an abuser is less likely to obtain a rapid “high.”

In certain preferred embodiments, when the dosage form is tampered with and exposed to a small amount (e.g., less than about 10 ml) of an embolizing liquid suitable for injection, the dosage form will be partially or fully solubilized or dissolved. Upon the addition of the embolizing liquid, and upon injection or inhalation of the dosage form exposed to the liquid, the tampered dosage containing form preferably becomes solidified or semi-solidified due to diffusion of the embolizing fluid into the body, temperature differences between the body and the solubilized embolic agent, and/or pH differences between the body and the embolizing agent, any of which will cause precipitation or coagulation of the embolizing agent, rendering the site of administration unsuitable for injection or snorting.

In certain embodiments the embolizing agent is not soluble in the G.I. tract environment but activates the biological process of platelet adhesion or blood coagulation when the dosage form is tampered with and subsequently injected or insufflated.

In certain embodiments of the present invention the formulation is not soluble in the aqueous environment at pH higher than 5 but is soluble or dispersible in the gastrointestinal tract at pH 1-5 to rapidly release the loaded API.

In certain embodiments, the embolizing agent is present in such an amount in the dosage form that attempts at evaporation (by the application of heat) to liquid mixture of the dosage form, such as in an effort to produce a higher concentration of the API, produces a semi-solid substance unsuitable for injection due to attainment of the liquification temperature required by the embolizing agent.

In certain embodiments of the present invention, the formulation is not soluble in the aqueous environment but is partially soluble or dispersible in the gastrointestinal tract at pH 1-5, then partially soluble or dispersible along moving through the small intestine (pH 5-7) to gradually release the loaded API.

When nasally insufflating the tampered dosage form, the embolizing agent can precipitate and become solid or semi-solid, such as following administration to the nasal passages due to the diffusion of the embolizing liquid into the body. This also makes such formulations less subject to nasal administration, as the precipitated embolic agent will prevent and/or reduce absorption of the abusable drug.

In certain preferred embodiments, the dosage forms are controlled release oral dosage forms comprising a therapeutically effective amount of an opioid analgesic with one or more of the embolizing agents such that the dosage form provides effective pain relief for at least about 12 hours, or at least about 24 hours when orally administered to a human patient.

In certain embodiments of the present invention the embolizing agent present in the dosage form is present in a substantially non-releasable form (i.e., “sequestered”) when the dosage form is administered intact as directed. Preferably, because the embolic agent is present in the dosage form in a substantially non-releasable form, it is not substantially released in the gastrointestinal tract when the dosage form is orally administered intact.

In other embodiments, the embolizing agent may not be “sequestered” as disclosed above wherein the embolizing agent is not released or minimally released from an intact dosage form, but may have a modified or sustained release so as not to dump the embolizing agent in a particular section of the gastrointestinal tract, e.g. the colon, where it may cause an unwanted effect such as bolus or blockage. The embolizing agent can be combined with an enteric carrier to delay its release or combined with a carrier to provide a sustained release of the embolizing agent. However, it is contemplated in the present invention that the embolizing agent will preferably not have any significant side effect (e.g., gastrointestinal side effect) even if all of the embolizing agent is immediately released upon oral administration of an intact dosage form as directed.

The embolizing agent(s) can also be in the dosage form in both a releasable form and a non-releasable form in any combination. For example, a dosage form can have an embolizing agent in releasable form and non-releasable form as disclosed in Sackler, U.S. Pat. No. 7,157,103, the disclosure of which is hereby incorporated by reference in its entirety.

Examples of drugs which may be susceptible to abuse and tampering and are suitable for the present invention include CNS depressants such as barbiturates and natural or synthetic opioids like morphine, oxycodone, hydrocodone, and codeine; anxiolytic agents such as benzodiazepene drugs; stimulants such as amphetamines; and locally acting anesthetic agents such as cocaine and lidocaine. Other examples of such drugs include cardiovascular drugs, respiratory drugs, such as pseudoephedrine and dextromethorphan, sympathomimetic drugs, cholinomimetic drugs, adrenergic drugs, antimuscarinic and antispasmodic drugs, skeletal muscle relaxants, diuretic drugs, anti-migraine drugs, anesthetics, sedatives and hypnotics, antiepileptics, psychopharmacologic agents, antipyretics, CNS stimulants, antineoplastic and immunosuppressive drugs, antimicrobial drugs, antihistamines, anti-inflammatories, antibiotics, decongestants, cough suppressants, and expectorants.

Analgesics, including opioid and non-opioid analgesics, are an important class of drug. Examples of analgesic drugs include alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethyl-thiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacyl-morphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papavereturn, pentazocine, phena-doxone, phenomorphan, phenazocine, phenoperidine, piminodine, piritramide, propheptazine, promedol, properidine, propoxyphene, sufentanil, tilidine, and tramadol.

The dosage form of the invention may optionally include one or more additional drugs that may or may not be in association with an embolic agent composition and the release of which additional drug may or may not be affected if the dosage form is tampered with. Such additional drugs may include those that are used in combination with a drug but which are not, in themselves, prone to being abused. An example of such an additional drug is guaifenesin or dextromethorphan, which is often used as a component of respiratory medications.

An embolizing agent and a drug may be in physical association as part of a dosage form or they may exist as separate and distinct elements of a dosage form. Because the release of a drug from a dosage form or the ability to fully abuse the dosage form is prevented, reduced or retarded upon injection or insufflation, the invention reduces or eliminates the sensation of euphoria and “attractiveness” that would otherwise be obtained by an individual who illicitly tampers with the dosage form in order to abuse a drug.

The dosage form of the invention provides a release of drug intended by the manufacturer of the dosage form when administered in an untampered form. However, when the dosage form is tampered with and injected and/or insufflated, the embolizing agent will precipitate or coagulate and retard or reduce the ability to further inhale or inject the dosage form as compared to that from a similar dosage form not containing the embolizing agent.

In another embodiment, the invention is a method for making a dosage form. In accordance with this embodiment of the invention, a drug is combined with an embolizing agent, either as one subunit or as separate drug and embolizing agent subunits, to obtain a dosage form from which the release rate of the drug is not significantly impeded by the presence of the embolizing agent if the dosage form has not been tampered with, but from which the release of the drug from the dosage form is reduced or retarded if the dosage form has been tampered with and injected and/or insufflated.

In another embodiment, the invention is a method for reducing or retarding the release of a drug from a dosage form that has been tampered with and injected and/or insufflated. According to this embodiment of the invention, an embolizing agent is combined with a dosage form containing a drug, wherein the embolizing agent does not significantly impede release of the drug from the dosage form when the dosage form has not been tampered with and is administered in a manner intended by the manufacturer but which the presence of the embolizing agent prevents, reduces or retards the ability of the potential abuser to fully insufflate or inject the drug from the dosage form if the dosage form has been tampered with.

In another embodiment, the invention is a method for administering a drug. In accordance with this embodiment of the invention, a dosage form containing an embolizing agent and a drug is administered to an individual in need of the drug, wherein the embolizing agent does not significantly impede the rate of release of the drug from the dosage form when the dosage form has not been tampered with but retards or reduces the release of the drug from the dosage form if the dosage form has been tampered with and injected and/or insufflated.

Preferred embolizing agents for the vehicle of the invention are those that are environmentally or biologically compatible with the digestive tract, have low or no toxicity in the dose called for in the application, and are compatible with the drug utilized. In certain embodiments, preferred embolizing agents of the invention are those agents that are soluble in the pH range of the G.I. tract but insoluble or partially insoluble in the pH range of the blood stream or the interstitial fluid. Preferably the embolizing agent act quickly at the site of administration as to not cause harm to the abuser.

It can be appreciated by those in the art that if an attempt is made to solubilize the embolizing agent with a solvent in which the embolizing agent is soluble or suspendable and then the resulting solution or suspension is administered either via injection or insufflation, the embolizing agent will precipitate at the site of administration. In the case of insufflation or injection, the embolizing fluid will diffuse from the site of administration and the embolizing agent will precipitate upon exposure with the blood, interstitial fluid, rectal fluid or nasal passage pH which is approximately pH 6.3-7.4. However an embolizing agent that is soluble in the pH of the G.I. tract will dissolve and not interfere with the delivery of the drug to the subject who is administering the dosage form as intended by the manufacturer.

Any concentration of the embolizing agent(s) may be used so long as the embolizing agent is compatible with the dosage form in the concentration used, is non-toxic in the dosage form when used as intended by the manufacturer, and forms an embolism at the non-oral site of administration.

Suitable examples of embolic agents are thrombin, cellulose diacetate polymer, albumin, gelatin, fibrinogen, lactoglobulin, immunoglobulin, actin, and acrylamide, a homopolymer or copolymer of an N-substituted (meth) acrylamide monomer as described by Ito in U.S. Pat. No. 5,525,334, polyacrylonitrile, polyurethane, polyvinylacetate, copolymer of an [meth-]acrylamide derivative and a hydrophilic comonomer as described by Gutowska, U.S. Pat. No. 6,979,464, nitrocellulose and copolymers of urethane/carbonate and copolymers of styrene/maleic acid and pH sensitive polymers consisting of copolymers of methyl and butyl methacrylate and dimethylaminoethyl methacrylates (amino methacrylates copolymer) manufactured as under the trademark Eudragit E by Evonik Industries (Piscataway, N.J.). The most preferred embolizing agents are those agents that are solid or semi-solid at the body temperature 98.6 C and an aqueous pH of 6.4-7.4. Other preferable pH sensitive polymers that are suitable for embolizing agents include Methacrylic Acid Copolymer Dispersion sold under the trade name EUDRAGIT® L 30 D-55 methacrylic acid copolymer, Type B sold under the trade names EUDRAGIT® S 100 and EUDRAGIT® S 12,5, Methacrylic Acid Copolymer, Type A sold under the brand name EUDRAGIT® L 100 and EUDRAGIT® L 12,5 and methacrylic acid copolymer, Type C sold under the brand name EUDRAGIT® L100-55 all by Evonik Industries (Piscataway, N.J.), Cellulose acetate trimellitate (CAT) (soluble when pH is higher than 4.8) Polyvinyl acetate phthalate (PVAP) (soluble when pH is higher than 5.0), hydroxypropyl methylcellulose phthalate (HPMCP) (soluble when pH is higher than 5.0-5.5), Eastacryl®30D (soluble when pH is higher than 5.5); Methacrylic acid/ethyl acrylate copolymers sold under the tradename Kollicoat®MAE 30 DP (soluble when pH is higher than 5.5); ; ACRYL-EZE® (soluble when pH is higher than 5.5); ACRYL-EZE MP® (soluble when pH is higher than 5.5), hydroxypropyl methylcellulose succinate (HPMCAS)-L (soluble when pH is higher than 5.0), hydroxypropyl methylcellulose succinate (HPMCAS)-M (soluble when pH is higher than 5.5), hydroxypropyl methylcellulose succinate (HPMCAS)-H (soluble when pH is higher than 6.0 to 6.5), cellulose acetate phthalate (CAP)) (soluble when pH is higher than 6.0).

If desired, a combination of pH sensitive embolizing agents may be used to form an embolus when a dosage form is tampered with.

The drug may be present in any physical state, such as a solid, a liquid, or a semisolid. Solid drug may be crystalline or amorphous, or a combination thereof. Such solid drug may be granulated with or without added excipients, and may be encapsulated in a material such as a polymer and/or a wax. A solid drug may also be in the form of a matrix in which the drug is distributed therein. A liquid drug may be granulated, such as by absorption to a solid substrate, or encapsulated with a suitable solid, such as a polymer or wax or combination thereof or blended with the embolizing agent in its liquid state.

The dosage form of the invention may be any oral dosage form by which a drug may be administered to an individual in order to achieve a desired pharmacological effect in that individual. The dosage form may be an oral dosage form, such as a tablet, a capsule containing a plurality of particles such as granules, a hard or soft-gel capsule containing a liquid, semi-solid such as a paste, or gel, a troche, a lozenge, a sachet, or a powder.

The dosage form of the invention may be one that provides immediate release of the drug when administered in the manner intended by the manufacturer or may be one that allows for controlled release of the drug. Because the danger of abuse of a drug due to illicit tampering of a dosage form containing the drug is highest with controlled release dosage forms, in a preferred embodiment, the dosage form of the invention is one that allows for controlled release of a drug.

The drug within the dosage form of the invention may be in the form of a multiplicity of subunits. Examples of subunits include particles, granules, microcapsules, microtablets, spheroids, beads, and rods. The subunits may or may not be individually or collectively covered with a coating composition which acts as a barrier or which modifies the release rate of the drug. Such coatings are typically made of one or more film-forming polymers. Examples of such polymers include acrylic polymers, cellulosic polymers, polylactic acid polymers, polyglycolic acid polymers, and co-polymers of polylactic and polyglycolic acid.

The drug of the dosage form may be free within the dosage form or may be in association with a material to form an “embolic agent composition”. As used herein, the term “embolic agent composition” refers to a physical or chemical association of a drug, such as a drug which may be susceptible to abuse and tampering, with an embolizing agent in a dosage form. As examples, the association of the drug with the embolic agent may be a distribution of the drug upon or within the embolic agent or an interaction or complexation of a drug with an embolic agent.

For example, a drug may be distributed upon or within a matrix, which may be a multiparticulate, layered matrix or semi-solid. The embolic agent composition, such as a matrix, may or may not be in the form of a multiplicity of subunits, which subunits may be in the form of particles, granules, microcapsules, microtablets, spheroids, beads, and rods in their solid, liquid or semi-solid form. The embolic agent composition may be in the form of one or more layers, such as within a solid tablet or one or more compartments in a multi-compartment capsule. The embolic agent composition, such as the matrix or the matrix-containing subunits or the one or more layers, may be individually or collectively covered with a coating composition. Coatings for control and release modification of drugs are known in the art. Semi-solid and liquid compositions are described herein.

The embolizing agent in the dosage form may be in the form of a solid, a semi-solid, or a liquid. The embolizing agent may be in the same or a different form as the embolic agent composition or the drug. The forms of a semi-solid or liquid embolizing agent are especially useful in capsule dosage forms, such as a soft or hard gelatin capsule. It may also be desirable to solubilize or suspend a solid form of embolizing agent in order to manufacture a liquid containing dosage form such as a soft or hard capsule.

In a preferred embodiment, an oral dosage form contains an amount of embolizing agent which makes a dosage form undesirable for injection and/or insufflation but which is effective when used in the fashion for the manufacturer intended.

In certain embodiments of the present invention the formulation is not soluble in an aqueous environment but is soluble or dispersible in the stomach compartment (pH 1-4) to rapidly release the loaded API.

In a preferred embodiment, a solid form embolizing agent is combined with an embolizing solvent or plasticizer to partially or fully dissolve the embolizing agent prior to combination with a drug.

Solid forms of embolizing agent include microcapsules, powders, and beads. Methods of preparing microcapsules, powders, and particle beads are well known in the art and include spray drying, spray chilling, rotary disk atomization, fluid bed coating, stationary nozzle coextrusion, extrusion-spheronization, hot-melt extrusion, centrifugal head coextrusion, and submerged nozzle coextrusion, phase separation, solvent evaporation, solvent extraction, interfacial polymerization, simple and complex coacervation, in-situ polymerization, hot melt extrusion and spheronization and liposomal encapsulation. Solid dosage forms include single or multilayered tablets, powders and soft or hardshell capsules.

The dosage form may contain additional optional components that may or may not be associated with a drug and/or an embolic agent composition. One optional component is one or more coatings that may be on the drug or on the embolic agent composition. These coatings, which act as barriers or modify the release rate of the coated substance, are made of film-forming polymers such as acrylic polymers, cellulosic polymers, polylactic acid polymers, polyglycolic acid polymers, and co-polymers of polylactic and polyglycolic acid. A plasticizer, such as acetyl triethyl citrate, acetyl tributyl citrate, triethyl citrate, diethyl phthalate, dibutyl phthalate, or dibutyl sebacate may be admixed with the polymer of the coating. Coatings on the embolic agent composition may be water permeable or impermeable. The embolic agent may be barrier coated so that these components will not interfere with the intended release profile from the drug or drug composition unless the dosage form is tampered with such as in the case with sequestered embolic agents. Coatings that are insoluble in the gastrointestinal tract are preferred, especially for coatings to be used for the embolic agent. In this way, the embolic agent does not function to inhibit release of the drug when the dosage form is administered as intended by the manufacturer, such as by swallowing the dosage form in its entirety. If desired, the dosage form may be covered with an enteric coating. Methods of making polymer coatings and of coating components of dosage forms are known in the art. The embolic agent may also not be barrier coated but be separated physically from the drug in the dosage form. In a preferred embodiment, the drug is separated from the embolic agent in the dosage form but the embolic agent is not coated. A preferred example of this embodiment would be a multilayered tablet in which a layer of the tablet would contain an embolic agent without drug and in a releasable form and a separate layer would contain the drug in either immediate or sustained release form.

An embolizing agent in substantially non-releasable form or sequestered form may be prepared by such methods as described by Oshlack, U.S. Pat. No. 6,696,088 in forming an opioid antagonist in substantially non-releasable form.

In certain embodiments of the present invention, an embolizing agent in a substantially non-releasable form may be prepared by combining the embolizing agent with one or more pharmaceutically acceptable hydrophobic materials. For example, embolizing agent particles may be coated with coating that substantially prevents the release of the embolizing agent, the coating comprising the hydrophobic materials(s). Another example would be an embolizing agent that is dispersed in a matrix that renders the embolizing agent substantially non-releasable, the matrix comprising the hydrophobic materials(s). In certain embodiments, the pharmaceutically acceptable hydrophobic material comprises a cellulose polymer selected from the group consisting of ethylcellulose, cellulose acetate, cellulose propionate (lower, medium or higher molecular weight), cellulose acetate propionate, cellulose acetate butyrate, cellulose acetate phthalate and cellulose triacetate. An example of ethylcellulose is one that has an ethoxy content of 44 to 55%. Ethylcellulose may be used in the form of an alcoholic solution. In certain other embodiments, the hydrophobic material comprises polylactic acid, polyglycolic acid or a co-polymer of the polylactic and polyglycolic acid.

In certain embodiments, the hydrophobic material may comprise a cellulose polymer selected from the group consisting of cellulose ether, cellulose ester, cellulose ester ether, and cellulose. The cellulosic polymers have a degree of substitution, D.S., on the anhydroglucose unit, from greater than zero and up to 3 inclusive. By degree of substitution is meant the average number of hydroxyl groups present on the anhydroglucose unit comprising the cellulose polymer that are replaced by a substituting group. Representative materials include a polymer selected from the group consisting of cellulose acylate, cellulose diacylate, cellulose triacylate, cellulose acetate, cellulose diacetate, cellulose triacetate, mono, di, and tricellulose alkanylates, mono, di, and tricellulose aroylates, and mono, di, and tricellulose alkenylates. Exemplary polymers include cellulose acetate having a D.S. and an acetyl content up to 21%; cellulose acetate having an acetyl content up to 32 to 39.8%; cellulose acetate having a D.S. of 1 to 2 and an acetyl content of 21 to 35%; cellulose acetate having a D.S. of 2 to 3 and an acetyl content of 35 to 44.8%.

More specific cellulosic polymers include cellulose propionate having a D.S. of 1.8 and a propyl content of 39.2 to 45 and a hydroxyl content of 2.8 to 5.4%; cellulose acetate butyrate having a D.S. of 1.8, an acetyl content of 13 to 15% and a butyryl content of 34 to 39%; cellulose acetate butyrate having an acetyl content of 2 to 29%, a butyryl content of 17 to 53% and a hydroxyl content of 0.5 to 4.7%; cellulose triacylate having a D.S. of 2.9 to 3 such as cellulose triacetate, cellulose trivalerate, cellulose trilaurate, cellulose tripalmitate, cellulose trisuccinate, and cellulose trioctanoate; cellulose diacylates having a D.S. of 2.2 to 2.6 such as cellulose disuccinate, cellulose dipalmitate, cellulose dioctanoate, cellulose dipentanoate, and coesters of cellulose such as cellulose acetate butyrate, cellulose acetate octanoate butyrate and cellulose acetate propionate.

Additional cellulose polymers useful for preparing an embolizing agent in a substantially non-releasable form include acetaldehyde dimethyl cellulose acetate, cellulose acetate ethylcarbamate, cellulose acetate methylcarbamate, and cellulose acetate dimethylaminocellulose acetate.

Acrylic polymers useful for preparation of the embolizing agent in a substantially non-releasable form include, but are not limited to, acrylic resins comprising copolymers synthesized from acrylic and methacrylic acid esters (e.g., the copolymer of acrylic acid lower alkyl ester and methacrylic acid lower alkyl ester) containing about 0.02 to 0.03 mole of a tri (lower alkyl) ammonium group per mole of the acrylic and methacrylic monomers used. An example of a suitable acrylic resin is a polymer manufactured by Rohm Pharma GmbH and sold under the Eudragit® RS. Eudragit RS30D is a preferred acrylic polymer. Eudragit® RS is a water insoluble copolymer of ethyl acrylate (EA), methyl methacrylate (MM) and trimethylammoniumethyl methacrylate chloride (TAM) in which the molar ratio of TAM to the remaining components (EA and MM) is 1:40. Acrylic resins such as Eudragit® RS may be used in the form of an aqueous suspension.

In certain embodiments of the invention, the acrylic polymer may be selected from the group consisting of acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate copolymers.

When the embolizing agent in a substantially non-releasable form comprises embolizing agent particles coated with a coating that renders the embolizing agent substantially non-releasable, and when a cellulose polymer or an acrylic polymer is used for preparation of the coating composition, a suitable plasticizer, e.g., acetyl triethyl citrate and/or acetyl tributyl citrate, may also be admixed with the polymer. The coating may also contain additives such as coloring agents, talc and/or magnesium stearate, which are well known in the coating art.

The coating composition may be applied onto the embolizing agent particles by spraying it onto the particles using any suitable spray equipment known in the art. For example, a Wurster fluidized-bed system may be used in which an air jet, injected from underneath, fluidizes the coated material and effects drying while the insoluble polymer coating is sprayed on. The thickness of the coating will depend on the characteristics of the particular coating composition being used. However, it is well within the ability of one skilled in the art to determine by routine experimentation the optimum thickness of a particular coating required for a particular dosage form of the present invention.

The pharmaceutically acceptable hydrophobic material useful for preparing an embolizing agent in a substantially non-releasable form includes a biodegradable polymer comprising a poly(lactic/glycolic acid) (“PLGA”), a polylactide, a polyglycolide, a polyanhydride, a polyorthoester, polycaprolactones, polyphosphazenes, polysaccharides, proteinaceous polymers, polyesthers, polydioxanone, polygluconate, polylactic-acid-polyethylene oxide copolymers, poly(hydroxybutyrate), polyphosphoesther or mixtures or blends of any of these.

In certain embodiments, a biodegradable polymer comprises a poly(lactic/glycolic acid), a copolymer of lactic and glycolic acid, having molecular weight of about 2,000 to about 500,000 daltons. The ratio of lactic acid to glycolic acid is from about 100:0 to about 25:75, with the ratio of lactic acid to glycolic acid of 65:35 being preferred.

Once the embolizing agent in a substantially non-releasable form is prepared, it may be combined with a drug, along with conventional excipients known in the art, to prepare the oral dosage form of the present invention. The polymers and other ingredients above may also be utilized to formulate the embolizing agents to slow release or delay release as disclosed above.

In certain preferred embodiments of the invention, the oral dosage form is a capsule or a tablet. When being formulated as a tablet, the embolizing agent and drug may be combined with one or more inert, non-toxic pharmaceutical excipients which are suitable for the manufacture of tablets. Such excipients include, for example, an inert diluent such as lactose; granulating and disintegrating agents such as cornstarch; binding agents such as starch; and lubricating agents such as magnesium stearate.

The oral dosage form of the present invention may be formulated to provide immediate release of the opioid agonist contained therein. In other embodiments of the invention, however, the oral dosage form provides sustained-release of the drug.

In certain embodiments, the oral dosage forms providing sustained release of the drug may be prepared by admixing the embolizing agent in a substantially non-releasable form with the drug and desirable pharmaceutical excipients to provide a tablet, and then coating the tablet with a sustained-release tablet coating.

In certain embodiments of the invention, sustained release opioid agonist tablets may be prepared by admixing the substantially non-releasable form of an embolizing agent with an embolizing agent in a matrix that provides the tablets with sustained-releasing properties. Methods of manufacturing solid dosage forms are well known in the art and include matrix formulations of coated and layered beads or pellets, single or multi-layer tablets and capsules, extruded monolithic dosage form and osmotic dosage forms. Preferred methods of manufacturing and forms of solid dosage forms are described in Oshlack, U.S. Pat. No. 7,144,587; Flath, U.S. Patent Application Publication No. 2007/0269505; and Kumar, U.S. Pat. No. 7,201,920, each of which is incorporated herein in their entirety.

The drug subject to tampering in combination with one or more embolizing agents can be formulated as an immediate release formulation or controlled release oral formulation in any suitable tablet, coated tablet, or multiparticulate formulation, such as a capsule, known to those skilled in the art. The controlled release dosage form may include a controlled release material which is incorporated into a matrix along with the drug and the embolizing agent. In addition, the embolizing agent may be separate from the matrix, or incorporated into the matrix. The controlled release dosage form may optionally comprise particles containing or comprising the drug, wherein the particles have diameter from about 0.1 mm to about 2.5 mm, preferably from about 0.5 mm to about 2 mm. The embolizing agent may be incorporated into these particles, or may be incorporated into a tablet or capsule containing these particles. Additionally, the embolizing agent may be incorporated into these particles, or may be incorporated into a tablet or capsule containing these particles. In a preferred embodiment, the particles are film coated with a material that permits release of the drug at a controlled rate in an environment of use. The film coat is chosen so as to achieve, in combination with the other stated properties, a desired in-vitro release rate.

In certain embodiments, the dosage forms of the present invention comprise immediate release matrixes containing the drug and the embolizing agent.

### Coated Beads

In certain embodiments of the present invention a hydrophobic material is used to coat inert pharmaceutical beads such as nu pariel 18/20 beads comprising an drug, and a plurality of the resultant solid controlled release beads may thereafter be placed in a soft or hard capsule in an amount sufficient to provide an effective controlled release dose when ingested and contacted by an environmental fluid, e.g., gastric fluid or dissolution media. The beads comprising the drug may further comprise the embolizing agent and/or one or more embolizing agents, or one or more embolizing agents may be prepared as separate beads and then combined in a dosage form including the controlled release beads comprising a drug, or the embolizing agent and/or one or more embolizing agents may be mixed in the dosage form with the controlled release beads comprising the drug. In preferred embodiments where the drug and the embolizing agent are mixed in a capsule as different beads, the beads have an exact or similar appearance in order to deter an abuser from manually separating the beads prior to abuse in order to avoid the embolizing agent. In tablet dosage forms, the embolizing agent may be included as a distinct layer.

The controlled release bead formulations of the present invention slowly release the drug. e.g., when ingested and exposed to gastric fluids, and then to intestinal fluids. The controlled release profile of the formulations of the invention can be altered, for example, by varying the amount of overcoating with the hydrophobic material, altering the manner in which a plasticizer is added to the hydrophobic material, by varying the amount of plasticizer relative to hydrophobic material, by the inclusion of additional ingredients or excipients, by altering the method of manufacture, etc. The dissolution profile of the ultimate product may also be modified, for example, by increasing or decreasing the thickness of the retardant coating. Spheroids or beads coated with a drug are prepared, e.g., by dissolving the drug in water and then spraying the solution onto a substrate, for example, nu pariel 18/20 beads, using a Wurster insert. Thereafter, the embolizing agent is optionally added to the beads prior to coating. Optionally, additional ingredients are also added prior to coating the beads. For example, a product which includes hydroxypropylmethylcellulose, etc. (e.g., Opadry™ commercially available from Colorcon, Inc.) may be added to the solution and the solution mixed (e.g., for about 1 hour) prior to application of the same onto the beads. The resultant coated substrate, in this example beads, may then be optionally overcoated with a barrier agent, to separate the drug from the hydrophobic controlled release coating. An example of a suitable barrier agent is one which comprises hydroxypropylmethylcellulose. However, any film-former known in the art may be used. It is preferred that the barrier agent does not affect the dissolution rate of the final product.

The beads may then be overcoated with an aqueous dispersion of the hydrophobic material. The aqueous dispersion of hydrophobic material preferably further includes an effective amount of plasticizer, e.g. triethyl citrate. Pre-formulated aqueous dispersions of ethylcellulose, such as Aquacoat™ or Surelease™, may be used. If Surelease is used, it is not necessary to separately add a plasticizer.

Plasticized hydrophobic material may be applied onto the substrate comprising the drug by spraying using any suitable spray equipment known in the art. In a preferred method, a Wurster fluidized-bed system is used in which an air jet, injected from underneath, fluidizes the core material and effects drying while the acrylic polymer coating is sprayed on. A sufficient amount of the hydrophobic material to obtain a predetermined controlled release of said drug when the coated substrate is exposed to aqueous solutions, e.g. gastric fluid, is preferably applied, taking into account the physical characteristics of the drug, the manner of incorporation of the plasticizer, etc. After coating with the hydrophobic material, a further overcoat of a film-former, such as Opadry™, is optionally applied to the beads. This overcoat is provided, if at all, in order to substantially reduce agglomeration of the beads.

The release of the drug from the controlled release formulation of the present invention can be further influenced, i.e., adjusted to a desired rate, by the addition of one or more release-modifying agents, or by providing one or more passageways through the coating. The ratio of hydrophobic material to water soluble material is determined by, among other factors, the release rate required and the solubility characteristics of the materials selected.

The release-modifying agents which function as pore-formers may be organic or inorganic, and include materials that can be dissolved, extracted or leached from the coating in the environment of use. The pore-formers may comprise one or more hydrophilic materials such as hydroxypropylmethylcellulose.

The controlled release coatings of the present invention can also include erosion-promoting agents such as starch and gums.

The controlled release coatings of the present invention can also include materials useful for making microporous lamina in the environment of use, such as polycarbonates comprised of linear polyesters of carbonic acid in which carbonate groups reoccur in the polymer chain.

The release-modifying agent may also comprise a semi-permeable polymer.

In certain preferred embodiments, the release-modifying agent is selected from hydroxypropylmethylcellulose, lactose, metal stearates, and mixtures of any of the foregoing. The controlled release coatings of the present invention may also include an exit means comprising at least one passageway, orifice, or the like. The passageway may be formed by such methods as those disclosed in U.S. Pat. Nos. 3,845,770; 3,916,889; 4,063,064; and 4,088,864. The passageway can have any shape such as round, triangular, square, elliptical, irregular, etc.

### Matrix Formulations

In certain embodiments of the present invention, the sustained release formulation is achieved via a matrix optionally having a controlled release coating as set forth herein. The present invention may also utilize a sustained release matrix that affords in-vitro dissolution rates of the drug and or antagonist within desired ranges and releases the drug and/or antagonist in a pH-dependent or pH-independent manner.

A non-limiting list of suitable sustained-release materials which may be included in a sustained-release matrix according to the invention includes hydrophilic and/or hydrophobic materials, such as gums, cellulose ethers, acrylic resins, polymers of acrylic acid cross-linked with polyalkenyl ethers or divinyl glycol, also known by trade name Carbopol® (Lubrizol, Whitecliff, Ohio), polyethylene oxide, protein derived materials, waxes, shellac, and oils such as hydrogenated castor oil and hydrogenated vegetable oil. However, any pharmaceutically acceptable hydrophobic or hydrophilic sustained-release material which is capable of imparting sustained-release of the drug may be used in accordance with the present invention. Preferred sustained-release polymers include alkylcelluloses such as ethylcellulose, acrylic and methacrylic acid polymers and copolymers; and cellulose ethers, especially hydroxyalkylcelluloses (especially hydroxypropylmethylcellulose) and carboxyalkylcelluloses. Preferred acrylic and methacrylic acid polymers and copolymers include methyl methacrylate, methyl methacrylate copolymers, ethoxyethyl methacrylates, ethyl acrylate, trimethyl ammonioethyl methacrylate, cyanoethyl methacrylate, aminoalkyl methacrylate copolymer, poly(acrylic acid), poly(methacrylic acid), methacrylic acid alkylamine copolymer, poly(methylmethacrylate), poly(methacrylicacid) (anhydride), polymethacrylate, polyacrylamide, poly(methacrylic acid anhydride), and glycidyl methacrylate copolymers.

Certain preferred embodiments utilize mixtures of any of the foregoing sustained-release materials in the matrix of the invention.

The matrix also may include a binder. In such embodiments, the binder preferably contributes to the sustained-release of the oxycodone or pharmaceutically acceptable salt thereof from the sustained-release matrix.

If an additional hydrophobic binder material is included, it is preferably selected from natural and synthetic waxes, fatty acids, fatty alcohols, and mixtures of the same. Examples include beeswax, carnauba wax, stearic acid and stearyl alcohol. This list is not meant to be exclusive. In certain preferred embodiments, a combination of two or more hydrophobic binder materials are included in the matrix formulations.

Preferred hydrophobic binder materials which may be used in accordance with the present invention include digestible, long chain (C₈-C₅₀, especially C₁₂-C₄₀), substituted or unsubstituted hydrocarbons, such as fatty acids, fatty alcohols, glyceryl esters of fatty acids, mineral and vegetable oils, natural and synthetic waxes and polyalkylene glycols. Hydrocarbons having a melting point of between 25° and 90° C. are preferred. Of the long-chain hydrocarbon binder materials, fatty (aliphatic) alcohols are preferred in certain embodiments. The oral dosage form may contain up to 80% (by weight) of at least one digestible, long chain hydrocarbon.

In certain embodiments, the hydrophobic binder material may comprise natural or synthetic waxes, fatty alcohols (such as lauryl, myristyl, stearyl, cetyl or preferably cetostearyl alcohol), fatty acids, including but not limited to fatty acid esters, fatty acid glycerides (mono-, di-, and tri-glycerides), hydrogenated fats, hydrocarbons, normal waxes, stearic acid, stearyl alcohol and hydrophobic and hydrophilic materials having hydrocarbon backbones. Suitable waxes include, for example, beeswax, glycowax, castor wax and carnauba wax. For purposes of the present invention, a wax-like substance is defined as any material which is normally solid at room temperature and has a melting point of from about 30 to about 100° C. In certain preferred embodiments, the dosage form comprises a sustained release matrix comprising an drug; one or more embolizing agents; and at least one water soluble hydroxyalkyl cellulose, at least one C₁₂-C₃₆, preferably C₁₄-C₂₂, aliphatic alcohol and, optionally, at least one polyalkylene glycol. The hydroxyalkyl cellulose is preferably a hydroxy (C₁ to C₆) alkyl cellulose, such as hydroxypropylcellulose, hydroxypropylmethylcellulose and, especially, hydroxyethyl cellulose. The amount of the at least one hydroxyalkyl cellulose in the present oral dosage form may be determined, inter alia, by the precise rate of drug release required. The aliphatic alcohol may be, for example, lauryl alcohol, myristyl alcohol or stearyl alcohol. In particularly preferred embodiments of the present oral dosage form, however, the at least one aliphatic alcohol is cetyl alcohol or cetostearyl alcohol. The amount of the aliphatic alcohol in the present oral dosage form may be determined, as above, by the precise rate of drug release required. It may also depend on whether at least one polyalkylene glycol is present in or absent from the oral dosage form. In the absence of at least one polyalkylene glycol, the oral dosage form preferably contains between about 20% and about 50% (by wt) of the aliphatic alcohol. When a polyalkylene glycol is present in the oral dosage form, then the combined weight of the aliphatic alcohol and the polyalkylene glycol preferably constitutes between about 20% and about 50% (by wt) of the total dosage form.

In one preferred embodiment, the ratio of, e.g., the at least one hydroxyalkyl cellulose or acrylic resin to the at least one aliphatic alcohol/polyalkylene glycol determines, to a considerable extent, the release rate of the drug from the formulation. In certain embodiments, a ratio of the hydroxyalkyl cellulose to the aliphatic alcohol/polyalkylene glycol of between 1:1 and 1:4 is preferred, with a ratio of between 1:2 and 1:3 being particularly preferred.

In certain embodiments, the polyalkylene glycol may be, for example, polypropylene glycol, or polyethylene glycol which is preferred. The average molecular weight of the at least one polyalkylene glycol is preferably between 1,000 and 15,000, especially between 1,500 and 12,000.

Another suitable sustained-release matrix comprises an alkylcellulose (especially ethylcellulose), a C₁₂ to C₃₆ aliphatic alcohol and, optionally, a polyalkylene glycol. In addition to the above ingredients, a sustained-release matrix may also contain suitable quantities of other materials, e.g., diluents, lubricants, binders, granulating aids and glidants that are conventional in the pharmaceutical art.

In order to facilitate the preparation of a solid, sustained-release oral dosage form according to this invention there is provided, in a further aspect of the present invention, a process for the preparation of a solid, sustained-release oral dosage form according to the present invention comprising incorporating an drug in a sustained-release matrix. Incorporation in the matrix may be effected, for example, by:

(a) forming granules comprising at least one hydrophobic and/or hydrophilic material as set forth above (e.g., a water soluble hydroxyalkyl cellulose) together with the drug and at least one embolizing agent;

(b) mixing the at least one hydrophobic and/or hydrophilic material-containing granules with at least one C₁₂-C₃₆ aliphatic alcohol, and

(c) optionally, compressing and shaping the granules.

The granules may be formed by any of the procedures well-known to those skilled in the art of pharmaceutical formulation. For example, in one preferred method, the granules may be formed by wet granulating the hydroxyalkyl cellulose, drug and one or more embolizing agents with water. In a particularly preferred embodiment of this process, the amount of water added during the wet granulation step is preferably between 1.5 and 5 times, especially between 1.75 and 3.5 times, the dry weight of the drug. Optionally, the drug and/or the one or more embolizing agents are added extragranularly.

A sustained-release matrix can also be prepared by, e.g., melt-granulation or melt-extrusion techniques. Generally, melt-granulation techniques involve melting a normally solid hydrophobic binder material, e.g., a wax, and incorporating a powdered drug therein. To obtain a sustained release dosage form, it may be necessary to incorporate a hydrophobic sustained-release material, e.g. ethylcellulose or a water-insoluble acrylic polymer, into the molten wax hydrophobic binder material. Examples of sustained-release formulations prepared via melt-granulation techniques are found, e.g., in U.S. Pat. No. 4,861,598.

The additional hydrophobic binder material may comprise one or more water-insoluble wax-like thermoplastic substances possibly mixed with one or more wax-like thermoplastic substances being less hydrophobic than said one or more water-insoluble wax-like substances. In order to achieve sustained release, the individual wax-like substances in the formulation should be substantially non-degradable and insoluble in gastrointestinal fluids during the initial release phases. Useful water-insoluble wax-like binder substances may be those with a water-solubility that is lower than about 1:5,000 (w/w).

The preparation of a suitable melt-extruded matrix according to the present invention may, for example, include the steps of blending the drug and at least one embolizing agent, together with a sustained release material and preferably a binder material to obtain a homogeneous mixture. The homogeneous mixture is then heated to a temperature sufficient to at least soften the mixture sufficiently to extrude the same. The resulting homogeneous mixture is then extruded, e.g., using a twin-screw extruder, to form strands. The extrudate is preferably cooled and cut into multiparticulates by any means known in the art. The matrix multiparticulates are then divided into unit doses. The extrudate preferably has a diameter of from about 0.1 to about 5 mm and provides sustained release of the oxycodone or pharmaceutically acceptable salt thereof for a time period of at least about 24 hours.

An optional process for preparing the melt extruded formulations of the present invention includes directly metering into an extruder a hydrophobic sustained release material, the drug, one or more embolizing agents, and an optional binder material; heating the homogenous mixture; extruding the homogenous mixture to thereby form strands; cooling the strands containing the homogeneous mixture; cutting the strands into matrix multiparticulates having a size from about 0.1 mm to about 12 mm; and dividing said particles into unit doses. In this aspect of the invention, a relatively continuous manufacturing procedure is realized.

Optionally, one or more embolizing agents may be prepared as separate multiparticulates (without the drug) and thereafter the multiparticulates may be combined with multiparticulates comprising drug (without the embolizing agent and/or one or more embolizing agents) in a dosage form.

Plasticizers, such as those described above, may be included in melt-extruded matrices. The plasticizer is preferably included as from about 0.1 to about 30% by weight of the matrix. Other pharmaceutical excipients, e.g., talc, mono or poly saccharides, lubricants and the like may be included in the sustained release matrices of the present invention as desired. The amounts included will depend upon the desired characteristic to be achieved.

The diameter of the extruder aperture or exit port can be adjusted to vary the thickness of the extruded strands. Furthermore, the exit part of the extruder need not be round; it can be oblong, rectangular, etc. The exiting strands can be reduced to particles using a hot wire cutter, guillotine, etc.

A melt extruded matrix multiparticulate system can be, for example, in the form of granules, spheroids or pellets depending upon the extruder exit orifice. For purposes of the present invention, the terms “melt-extruded matrix multiparticulate(s)” and “melt-extruded matrix multiparticulate system(s)” and “melt-extruded matrix particles” shall refer to a plurality of units, preferably within a range of similar size and/or shape and containing one or more active agents and one or more excipients, preferably including a hydrophobic sustained release material as described herein. Preferably the melt-extruded matrix multiparticulates will be of a range of from about 0.1 to about 12 mm in length and have a diameter of from about 0.1 to about 5 mm. In addition, it is to be understood that the melt-extruded matrix multiparticulates can be any geometrical shape within this size range. In certain embodiments, the extrudate may simply be cut into desired lengths and divided into unit doses of the therapeutically active agent without the need of a spheronization step.

In one preferred embodiment, oral dosage forms are prepared that include an effective amount of melt-extruded matrix multiparticulates within a capsule. For example, a plurality of the melt-extruded matrix multiparticulates may be placed in a hard or soft capsule in an amount sufficient to provide an effective sustained release dose when ingested and contacted by gastrointestinal fluid.

In another embodiment, a suitable amount of the multiparticulate extrudate is compressed into an oral tablet using conventional tableting equipment using standard techniques. Techniques and compositions for making tablets (compressed and molded), capsules (hard and soft) and pills are also described in Remington's Pharmaceutical Sciences, (Arthur Osol, editor), 1553-1593 (1980).

In yet another preferred embodiment, the extrudate can be shaped into tablets as set forth in U.S. Pat. No. 4,957,681 (Klimesch, et. al.).

Optionally, the sustained-release matrix multiparticulate systems, tablets, or capsules can be coated with a sustained release coating such as the sustained release coatings described herein. Such coatings preferably include a sufficient amount of hydrophobic and/or hydrophilic sustained-release material to obtain a weight gain level from about 2 to about 25 percent, although the overcoat may be greater depending upon, e.g., the desired release rate. The coating can optionally contain one or more of the embolizing agents. In such embodiments, an optional second overcoat can be applied as to minimize the perception of the embolizing agent when a dosage form of the present inventions administered intact.

The dosage forms of the present invention may further include combinations of melt-extruded matrix multiparticulates containing a drug; and one or more embolizing agents. Furthermore, the dosage forms can also include an amount of an immediate release drug for prompt therapeutic effect. The immediate release drug may be incorporated, e.g., as separate multiparticulates within a gelatin capsule, or may be coated on the surface of, e.g., melt extruded matrix multiparticulates.

The sustained-release profile of the melt-extruded formulations of the invention can be altered, for example, by varying the amount of sustained-release material, by varying the amount of plasticizer relative to other matrix constituents, by varying the amount of hydrophobic material, by the inclusion of additional ingredients or excipients, by altering the method of manufacture, etc.

In other embodiments of the invention, melt-extruded formulations are prepared without the inclusion of the drug and one or more embolizing agents; which is added thereafter to the extrudate. Such formulations typically will have the drug and one or more embolizing agents blended together with the extruded matrix material, and then the mixture would be tableted in order to provide a slow release formulation. Such formulations may be advantageous, for example, when the drug and one or more embolizing agents included in the formulation is sensitive to temperatures needed for softening the hydrophobic material and/or the retardant material.

Typical melt-extrusion production systems suitable for use in accordance with the present invention include a suitable extruder drive motor having variable speed and constant torque control, start-stop controls, and a meter. In addition, the production system will include a temperature control console, which includes temperature sensors, cooling means and temperature indicators throughout the length of the extruder. In addition, the production system will include an extruder such as a twin-screw extruder which consists of two counter-rotating intermeshing screws enclosed within a cylinder or barrel having an aperture or die at the exit thereof. The feed materials enter through a feed hopper and are moved through the barrel by the screws and are forced through the die into strands which are thereafter conveyed such as by a continuous movable belt to allow for cooling and being directed to a pelletizer or other suitable device to render the extruded ropes into the matrix multiparticulate system. Suitable apparatus will be apparent to those of ordinary skill in the art.

A further aspect of the invention is related to the preparation of melt-extruded matrix multiparticulates as set forth above in a manner which controls the amount of air included in the extruded product. By controlling the amount of air included in the extrudate, the release rate of the drug and one or more embolizing agents may be altered.

Thus, in a further aspect of the invention, the melt-extruded product is prepared in a manner which substantially excludes air during the extrusion phase of the process. This may be accomplished, for example, by using a Leistritz extruder having a vacuum attachment. The extruded matrix multiparticulates prepared according to the invention using the Leistritz extruder under vacuum provides a melt-extruded product having different physical characteristics. In particular, the extrudate is substantially non-porous when magnified, e.g., using a scanning electron microscope which provides an SEM (scanning electron micrograph). Such substantially non-porous formulations may provide a faster release of the therapeutically active agent, relative to the same formulation prepared without vacuum. SEMs of the matrix multiparticulates prepared using an extruder under vacuum appear very smooth, and the multiparticulates tend to be more robust than those multiparticulates prepared without vacuum. It has been observed that in at least certain formulations, the use of extrusion under vacuum provides an extruded matrix multiparticulate product which is more pH-dependent than its counterpart formulation prepared without vacuum.

Alternatively, the melt-extruded product is prepared using a Werner-Pfleiderer twin screw extruder.

In certain embodiments, a spheronizing agent is added to a granulate or matrix multiparticulate and then spheronized to produce sustained release spheroids. The spheroids are then optionally overcoated with a sustained release coating by methods such as those described above.

Spheronizing agents which may be used to prepare the matrix multiparticulate formulations of the present invention include any art-known spheronizing agent. Cellulose derivatives are preferred, and microcrystalline cellulose is especially preferred. A suitable microcrystalline cellulose is, for example, the material sold as Avicel PH 101 (TradeMark, FMC Corporation). The spheronizing agent is preferably included as about 1 to about 99% of the matrix multiparticulate by weight.

In certain embodiments, in addition to the drug, one or more embolizing agents, and spheronizing agent, the spheroids may also contain a binder. Suitable binders, such as low viscosity, water soluble polymers, will be well known to those skilled in the pharmaceutical art. However, water soluble hydroxy lower alkyl cellulose, such as hydroxy propyl cellulose, are preferred. Additionally (or alternatively) the spheroids may contain a water insoluble polymer, especially an acrylic polymer, an acrylic copolymer, such as a methacrylic acid-ethyl acrylate copolymer, or ethyl cellulose.

In certain embodiments, a sustained release coating is applied to the sustained release spheroids, granules, or matrix multiparticulates. In such embodiments, the sustained-release coating may include a water insoluble material such as (a) a wax, either alone or in admixture with a fatty alcohol; or (b) shellac or zein. The coating is preferably derived from an aqueous dispersion of the hydrophobic sustained release material.

In certain embodiments, it is necessary to overcoat the sustained release spheroids, granules, or matrix multiparticulates comprising the drug and one or more embolizing agents, and sustained release carrier with a sufficient amount of the aqueous dispersion of, e.g., alkylcellulose or acrylic polymer, to obtain a weight gain level from about 2 to about 50%, e.g., about 2 to about 25%, in order to obtain a sustained-release formulation. The overcoat may be lesser or greater depending upon, e.g., the desired release rate, the inclusion of plasticizer in the aqueous dispersion and the manner of incorporation of the same. Cellulosic materials and polymers, including alkylcelluloses, are sustained release materials well suited for coating the sustained release spheroids, granules, or matrix multiparticulates according to the invention. Simply by way of example, one preferred alkylcellulosic polymer is ethylcellulose, although the artisan will appreciate that other cellulose and/or alkylcellulose polymers may be readily employed, singly or in any combination, as all or part of a hydrophobic coating according to the invention.

One commercially available aqueous dispersion of ethylcellulose is Aquacoat™(FMC Corp., Philadelphia, Pa., U.S.A.). Aquacoat™ is prepared by dissolving the ethylcellulose in a water-immiscible organic solvent and then emulsifying the same in water in the presence of a surfactant and a stabilizer. After homogenization to generate submicron droplets, the organic solvent is evaporated under vacuum to form a pseudolatex. The plasticizer is not incorporated in the pseudolatex during the manufacturing phase. Thus, prior to using the same as a coating, it is necessary to intimately mix the Aquacoat™ with a suitable plasticizer prior to use.

Another aqueous dispersion of ethylcellulose is commercially available as Surelease™ (Colorcon, Inc., West Point, Pa., U.S.A.). This product is prepared by incorporating plasticizer into the dispersion during the manufacturing process. A hot melt of a polymer, plasticizer (dibutyl sebacate), and stabilizer (oleic acid) is prepared as a homogeneous mixture, which is then diluted with an alkaline solution to obtain an aqueous dispersion which can be applied directly to the sustained release spheroids, granules, or matrix multiparticulates.

In other preferred embodiments of the present invention, the sustained release material comprising the sustained-release coating is a pharmaceutically acceptable acrylic polymer, including but not limited to acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate copolymers.

In certain preferred embodiments, the acrylic polymer is comprised of one or more ammonio methacrylate copolymers. Ammonio methacrylate copolymers are well known in the art, and are described in the National Formulary (NF) XVII as fully polymerized copolymers of acrylic and methacrylic acid esters with a low content of quaternary ammonium groups. In order to obtain a desirable dissolution profile, it may be necessary to incorporate two or more ammonio methacrylate copolymers having differing physical properties, such as different molar ratios of the quaternary ammonium groups to the neutral (meth)acrylic esters.

Certain methacrylic acid ester-type polymers are useful for preparing pH-dependent coatings which may be used in accordance with the present invention. For example, there are a family of copolymers synthesized from diethylaminoethyl methacrylate and other neutral methacrylic esters, also known as methacrylic acid copolymer or polymeric methacrylates, commercially available as Eudragit™ from Evonik Degussa, Darmstadt, Germany. There are several different types of Eudragit™. For example, Eudragit E is an example of a methacrylic acid copolymer which dissolves in acidic media. Eudragit L is a methacrylic acid copolymer which does not swell at about pH<5.7 and is soluble at about pH>6. Eudragit S does not swell at about pH<6.5 and is soluble at about pH>7. Eudragit RL and Eudragit RS are water swellable, and the amount of water absorbed by these polymers is pH-dependent; however, release from the dosage forms coated with Eudragit RL and RS are pH-independent. In certain preferred embodiments, the acrylic coating comprises a mixture of two acrylic resin lacquers commercially available from Rohm under the Trade names Eudragit™ RL30D and Eudragit™ RS30D, respectively. Eudragit™ RL30D and Eudragit™ RS30D are copolymers of acrylic and methacrylic esters with a low content of quaternary ammonium groups, the molar ratio of ammonium groups to the remaining neutral (meth)acrylic esters being 1:20 in Eudragit® RL30D and 1:40 in Eudragit® RS30D. The mean molecular weight is about 150,000. The code designations RL (high permeability) and RS (low permeability) refer to the permeability properties of these agents. Eudragit® RL/RS mixtures are insoluble in water and in digestive fluids. However, coatings formed from the same are swellable and permeable in aqueous solutions and digestive fluids.

The Eudragit® RL/RS dispersions of the present invention may be mixed together in any desired ratio in order to ultimately obtain a sustained-release formulation having a desirable dissolution profile. Desirable sustained-release formulations may be obtained, for instance, from a retardant coating derived from 100% Eudragit® RL, 50% Eudragit® RL and 50% Eudragit® RS, and 10% Eudragit® RL:Eudragit® 90% RS. Of course, one skilled in the art will recognize that other acrylic polymers may also be used, such as, for example, Eudragit® L. In embodiments of the present invention where the coating comprises an aqueous dispersion of a hydrophobic sustained release material, the inclusion of an effective amount of a plasticizer in the aqueous dispersion of hydrophobic material will further improve the physical properties of the sustained-release coating. For example, because ethylcellulose has a relatively high glass transition temperature and does not form flexible films under normal coating conditions, it is preferable to incorporate a plasticizer into an ethylcellulose coating containing sustained-release coating before using the same as a coating material. Generally, the amount of plasticizer included in a coating solution is based on the concentration of the film-former, e.g., most often from about 1 to about 50 percent by weight of the film-former. Concentration of the plasticizer, however, can only be properly determined after careful experimentation with the particular coating solution and method of application.

Examples of suitable plasticizers for ethylcellulose include water insoluble plasticizers such as dibutyl sebacate, diethyl phthalate, triethyl citrate, tributyl citrate, and triacetin, although it is possible that other water-insoluble plasticizers (such as acetylated monoglycerides, phthalate esters, castor oil, etc.) may be used. Triethyl citrate is an especially preferred plasticizer for the aqueous dispersions of ethyl cellulose of the present invention. Examples of suitable plasticizers for the acrylic polymers of the present invention include, but are not limited to citric acid esters such as triethyl citrate NF XVI, tributyl citrate, dibutyl phthalate, and possibly 1,2-propylene glycol. Other plasticizers which have proved to be suitable for enhancing the elasticity of the films formed from acrylic films such as Eudragit® RL/RS lacquer solutions include polyethylene glycols, propylene glycol, diethyl phthalate, castor oil, and triacetin. Triethyl citrate is an especially preferred plasticizer for the aqueous dispersions of ethyl cellulose of the present invention.

In certain embodiments, the uncoated/coated sustained release spheroids, granules, or matrix multiparticulates containing the drug and one or more embolizing agents; are cured until an endpoint is reached at which the sustained release spheroids, granules, or matrix multiparticulates provide a stable dissolution of the opioid. The curing endpoint may be determined by comparing the dissolution profile (curve) of the dosage form immediately after curing to the dissolution profile (curve) of the dosage form after exposure to accelerated storage conditions of, e.g., at least one month at a temperature of 40° C. and a relative humidity of 75%. Cured formulations are described in detail in U.S. Pat. Nos. 6,024,982.

In addition to the above ingredients, the spheroids, granules, or matrix multiparticulates may also contain suitable quantities of other materials, e.g., diluents, lubricants, binders, granulating aids, and glidants that are conventional in the pharmaceutical art in amounts up to about 50% by weight of the formulation if desired. The quantities of these additional materials will be sufficient to provide the desired effect to the desired formulation.

Specific examples of pharmaceutically acceptable carriers and excipients that may be used to formulate oral dosage forms are described in the Handbook of Pharmaceutical Excipients, American Pharmaceutical Association (1986), incorporated by reference herein.

The oral dosage form of the present invention may be a semi-solid. Semi-solids may include emulsions, gels, ointments, pastes or creams as defined in the United States Pharmacopeia 30 NF 25, Chapter 1151 (2007), First Supplement, herein incorporated by reference in its entirety. A preferred semi-solid dosage form is a gel.

The forms of semi-solid are especially useful to allow for content uniformity of the drug and the embolizing agent. A notable improvement in solubilizing a solid form of an embolizing agent is that the embolizing agent may be pre-dissolved so that when an illicit user adds an embolizing liquid, the embolizing agent may go into the embolizing liquid solution more rapidly or may be able to mask the existence of the embolizing agent to prevent the illicit user from identifying or separating the embolizing agent from the composition. Other improvements in semi-solid form are anticipated to be ease of manufacturing, drug and/or embolic agent or drug stability, increased bioavailability of the drug and a reduction in cross contamination from the drug in the manufacturing environment. Chakravorty, U.S. Pat. No. 7,056,530 describes the increased bioavailability of the use of an immunosuppression agent, hydrophilic agent, lipophilic agent, surfactants, antioxidant and preservative in a self-emulsifiable soft gelatin capsule liquid formulation for oral administration. Said composition exhibits enhanced bio-absorption and immunosuppression activities, and improved capability to release the drug in reduced time with reduced toxicity and variability that is inter and intra-patient bio-absorption variability. Rouffler, U.S. Pat. No. 6,221,391 describes a self-emulsifying solution of ibuprofen suitable for encapsulation into a soft gelatin capsule which exhibits decreased absorption time of the drug in the body compared to a non-liquefied dosage form and increased solubility of the ibuprofen which is normally practically insoluble in water or the acidic environment of the stomach.

The forms of a semi-solid are especially useful to be filled into capsule dosage forms, such as a soft or hard gelatin capsules. Hard and soft capsule manufacturing and forms such as gelatin or alternate materials such as hydroxypropyl methyl cellulose, carageenan and the like are well known in the art. Capsules may be prepared and tested by techniques well known in the art, for example, as described in Remington's Pharmaceutical Sciences, Mack Publishing Company, and especially in chapter 89, the pharmaceutical preparation and manufacture of “Tablets, Capsules and Pills.” Methods of preparing microcapsules are well known in the art and include but are not limited to rotary disk atomization, stationary nozzle coextrusion, centrifugal head coextrusion, and submerged nozzle coextrusion, extrusion-spheronization and in-situ polymerization.

In one embodiment, the invention is a method for making a composition or formulation, such as a pharmacologic formulation, by dissolving, dispersing, emulsifying, or suspending a drug in a vehicle containing an embolizing agent and an appropriate solvent, with or without suitable additives, and by utilizing an additional volatilized organic solvent as a processing aid. For example, it is a common practice in the wet granulation of pharmaceutical tablet production to utilize alcohol as a wetting and/or granulating agent. The alcohol is evaporated from the granulation mixture prior to the formation of tablets. In this case, the alcohol is functioning as a processing aid in order to pre-dissolve the embolizing agent but may be evaporated out of the final formation of the formulation. According to this embodiment of the invention, it may be desirable to incorporate a drug within the embolizing agent by using a volatilized organic solvent prior to or after combining a solvent with the embolizing agent. The volatilized solvent in this case is utilized as a processing aid and for incorporation of the drug and to enhance the interaction of the solvents and plasticizers and the embolizing agent. The volatilized solvent is removed from the final dosage form prior to application to the organism or environment and is not utilized as a component of the vehicle.

The composition of the invention, containing a drug dissolved, dispersed, emulsified, or suspended within an embolizing agent and solvents and/or plasticizers, may be encapsulated, such as within a capsule or within a coating. The capsule or coating compositions may or may not contain additional drug and may or may not contain a embolizing agent or an solvents and plasticizers. For example, soft gel or microcapsule compositions of the invention may be coated to provide mechanical stability, or to protect the embolizing agent-solvents and plasticizers vehicle or vehicle compositions from oxidation.

In a preferred embodiment of an oral semi-solid dosage form is an embolizing agent which is soluble between pH 1-5, are biologically compatible, have low toxicity in the levels utilized, may exist as a liquid, semi-solid or solid form, are and are compatible with a wide variety of additional additives and components. One or more suitable embolizing agent may be utilized within a vehicle to provide the desired release characteristics or processing requirements. In addition to one or more embolizing agent(s), the dosage form includes one or more non-volatilized solvents, plasticizers or suspending liquids (solvents and plasticizers). The solvents and plasticizers may completely or partially solubilize or plasticize just the embolizing agent, may completely or partially solubilize both the drug and the embolizing agent, may completely or partially solubilize or plasticize the embolizing agent, and/or suspend, dissolve or emulsify the drug, or may suspend, dissolve or emulsify the drug and the embolizing agent. The solvents and plasticizers may also act as a co-solvent or co-plasticizer to another solvents and plasticizers. The solvents and plasticizers may have variable water solubility or miscibility, a characteristic which may be used to further modulate the release characteristics of the drug from the vehicle. The solvent and plasticizer may act as an embolizing liquid without the addition of an external embolizing liquid by an illicit user.

Examples of preferred solvents and plasticizers include but are not limited to citric acid esters such as triethyl citrate (TEC), acetyl triethyl citrate (ATEC), tributyl citrate (TBC), acetyl tributyl citrate (ATBC), acetyltri-n-hexyl citrate (A-6), and butyryltri-triethyl n-hexyl citrate (B-6), lecithin, polyoxyethylene sorbitan fatty acid esters, fatty acid salts, mono and diacetyl tartaric acid esters of mono and diglycerides of edible fatty acids, citric acid esters of mono and diglycerides of edible fatty acids, saccharose esters of fatty acids, polyglycerol esters of fatty acids, polyglycerolesters of internal esterified castor oil acid, sodium stearoyllactylate, polyoxyethylated hydrogenated castor oil, block copolymers of ethylene oxide and propylene oxide, polyoxyethylene fatty alcohol ether, polyoxyethylene steraric acid ester, ethyl lactate, phthalates such as dimethyl phthalate (DMP), diethyl phthalate (DEP), dibutyl phthalate (DBP), dioctyl phthalate, glycol ethers such as ethylene glycol diethyl ether, propylene glycol monomethyl ether, PPG-2 myristyl ether propionate, diethylene glycol monoethyl ether, propylene glycol monotertiary butyl ether, dipropylene glycol monomethyl ether, N-methyl-2-pyrrolidone, 2 pyrrolidone, isopropyl myristate, isopropyl palmitate, octyl palmitate, dimethylacetamide, propylene glycol, propylene glycol monocaprylate, propylene glycol caprylate/caprate, propylene glycol monolaurate, glycerol, glycofurol, linoleic acid, linoeoyl macrogol-6 glycerides, oleic acid and esters such as glyceryl dioleate, ethyl oleate, oleoyl macrogol-6 glycerides, esters such as ethylbenzoate, benzyl benzoate, sucrose esters such as sucrose acetate isobutyrate, esters of lactic acid, esters of oleic acid, sebacates such as dimethyl sebacate, diethyl sebacate, dibutyl sebacate, dipropylene glycol methyl ether acetate (DPM acetate), propylene carbonate, propylene glycol laurate, dimethylsulfoxide, polyethylene glycol, dimethyl isosorbide, methylsulfonic acid, gamma butyrolactone, glycerol formal, soketal, ethanol, and water.

Other suitable solvents and plasticizers include oils, fats and their derivatives. Oils derived from animals or from plant seeds of nuts typically include glycerides of the fatty acids, chiefly oleic, palmitic, stearic, and linolenic. Non-limiting examples of suitable natural, semi-synthetic and synthetic oils include vegetable oil, peanut oil, medium chain triglycerides, soybean oil, almond oil, olive oil, sesame oil, peanut oil, fennel oil, camellia oil, corn oil, castor oil, cotton seed oil, peppermint oil, safflower oil and soybean oil, either crude or refined, and medium chain fatty acid triglycerides, mineral oils, suitable oil or fat, as for instance completely or partially hydrogenated vegetable oils or completely or partially hydrogenated animal fats, saturated polyglycolized glycerides, semi-synthetic glycerides, glyceryl esters of fatty acids, glyceryl behenate, glyceryl di and tri stearate, glyceryl palmitostearate, lauroyl macrogol-32 glycerides, stearoyl macrogol-32 glycerides, polyethylene glycol esters of fatty acids such as glyceryl laurate, PEG-32 glyceryl palmitostearate, PEG-32 glyceryl stearate, cetyl palmitate, stearyl alcohol, and cetyl alcohol.

Any suitable ratio of embolizing agent to solvent and plasticizers may be utilized to provide the desired release characteristics to the drug included therein so long as the combination of the embolizing agent and the solvents and plasticizers are sufficient to form a semi-solid as defined herein.

For example, the ratio of embolizing agent to solvents and plasticizers may be at any ratio % w/w between 99:1 and 1:99. Specific examples of ratios % w/w of embolizing agent to solvents and plasticizers that may be utilized include 5:95, 10:90, 20:80, 30:70, 40:60, 50:50, 60:40, 70:30, 80:20, 90:10. Preferred ratios are of ratios % w/w of embolizing agent to solvents and plasticizers include 5:95, 10:90, 20:80, 30:70, 40:60 and 50:50.

Generally, a composition that is hydrophilic will tend to release drug such as a drug loaded in the vehicle more rapidly than a similar formulation possessing hydrophobic properties. Also, a more viscous composition may release drug at a slower rate than a similar less viscous composition. Additionally, a vehicle constituting a embolizing agent and a solvent and/or plasticizer and combination of hydrophilic and hydrophobic components or additives may modulate the release characteristics of the dissolved, dispersed, emulsified, or suspended drug from the compositions.

Thus, the release characteristics and absorption of a drug may be modulated by the rheology and/or hydrophilicity or hydrophobicity of the composition containing the embolizing agent and a solvents and/or plasticizer. The release characteristics may be further modified by combining one or more suitable additives in or with the vehicle. These additives may include additional embolizing agents and solvents and/or plasticizers of varying physicochemical characteristics. The release characteristics and absorption of a drug may be further modified by modulating the ratio of embolizing agent to solvents and plasticizers and the amount of drug incorporated in the composition.

Thus, release of a drug from the dosage form containing an embolizing agent and solvents and plasticizers may be obtained by the use of one embolizing agent and one solvents and plasticizers as the vehicle or by combining one or more embolizing agents with different physical and chemical properties, one or more solvents and plasticizers with different physical and chemical properties, and/or by optionally combining one or more additives of different physical and chemical properties.

A preferred embodiment of the semi-solid dosage forms is a sustained release dosage form that contains a solubilized or suspended embolizing agent and a co-solubilized or suspended sustained releasing agent such as polymers of acrylic acid cross-linked with polyalkenyl ethers or divinyl glycol. Such co-solubilized or suspended sustained releasing agents are present in the formulation form 0.1% up to 45% w/w of the formulation, and most preferably 1% to 15% w/w of the formulation.

Another preferred embodiment is an immediate release dosage form containing a solubilized or suspended embolizing agent and a co-solubilized or suspended sustained releasing agent such as polymers of acrylic acid cross-linked with polyalkenyl ethers or divinyl glycol. Such co-solubilized or suspended sustained releasing agents are present in the formulation form 0.1% up to 45% w/w of the formulation, and most preferably 1% to 15% w/w of the formulation.

Additives may be included in the composition containing the vehicle of the invention in order to obtain the desired release characteristics of the drug. Liquid, semisolid, or solid additives may be added, either singly or in combination, to the semi-solid composition to modify the physicochemical as well as biological characteristics of the vehicle such as, hydrophilicity or hydrophobicity, consistency or viscosity, absorption rate and degradation rate at implantation or application sites, color, and stability. Addition of hydrophilic liquid, semisolid, or solid additives will increase the hydrophilicity of the semi-solid dosage forms prepared from blends of embolizing agent and solvents and plasticizers whereas, addition of hydrophobic liquid, semisolid or solid additives will increase the hydrophobicity of a semi-solid dosage forms prepared from blends of drug, embolizing agents, solvents and/or plasticizers. Hydrophilic vehicles may tend to be absorbed more rapidly or release the drug more rapidly than the hydrophobic vehicles from the site of administration, injection or application. Addition of semisolid and solid additives may increase the viscosity of the vehicles, which generally decreases the release rate of a drug as compared to addition of a liquid additive. Depending on the intended release rate and site of release in the gastrointestinal tract of the drug, the formulation may be altered to obtain the desired release characteristics for the drug. The amount of additive used will in general be a function of the nature of the additive and the effect to be achieved, and can be easily determined by the practitioner.

In a preferred embodiment, a semi-solid composition of the invention containing one or more drug and containing one or more embolizing agent and solvents and plasticizers may be prepared by any suitable blending or incorporation method. Typically, the embolizing agent is blended with the solvents and plasticizers until the embolizing agent is dissolved or suspended and the composition achieves a uniform consistency and desired rheological characteristics of a gel or semi-solid. The drug is then added either at the same or a higher or lower temperature and combined with the embolic agent, such as by blending or mixing until the drug is dissolved, or dispersed, emulsified, or suspended in the vehicle. The drug may be added at any stage in the preparation. For example, the drug may be combined with the embolizing agent before the solvents and plasticizers is combined, or may be combined with the solvent or plasticizer before the embolizing agent is combined, or may be combined with the embolizing agent and solvents and plasticizers following the combination of these components.

When present, the additive is typically present in the compositions in an amount ranging from about 0.1 percent to about 99 percent by weight, relative to the total weight of the composition, and more typically, is present in the composition in an amount ranging from about 1, 2, or 5 percent to about 40 percent by weight. Certain additives, such as buffers, are only present in small amounts in the composition while certain polymers may be present at higher levels, such as 20 to 30 percent

The following categories are non-limiting examples of classes of additives that may be employed in the composition. Given the disclosure herein and the objects to be achieved, one of skill in the art will easily know how to select other additives to achieve a desired purpose. All of these embodiments are considered to fall within the disclosed invention.

Additives components, such as carbohydrates, preservatives, stabilizers, anti-oxidants, coloring agents, isotonic agents, flavorings, humectants, sequesterants, vitamins and vitamin precursors, salts, surfactants, phospholipids, viscosity increasing agents and contrast agents or dyes may be added as desired. As preferred examples of carbohydrates are monosaccharides (simple sugars such as fructose and its isomer glucose (dextrose); disaccharides such as sucrose, maltose, cellobiose, lactose; starch; polysaccharides; polyols such as mannitol and sorbitol; dextrins such as maltodextrin, and cyclodextrins such as α-cyclodextrin, β-cyclodextrin, hydroxypropyl-β-cyclodextrin, and sulfobutylether-β-cyclodextrin. As preferred examples of preservatives, paraben are given with methyl paraben and propyl paraben given as most preferred preservatives. As preferred examples of anti-oxidants, butyl hydroxyanisole, butyl hydroxytoluene, propyl gallate, vitamin E acetate, and purified hydroquinone are given with vitamin E acetate and butyl hydroxytoluene given as most preferred anti-oxidants. Given as preferred examples of humectant is sorbitol. Given as preferred examples of flavorings are peppermint oil, spearmint oil, wintergreen oil, menthol and saccharin. Given as a preferred example of a sequesterant is citric acid. Preferred examples of vitamins are vitamin A, C, D and E and K, and vitamin E acetate. Examples of salts include aluminum salts, aluminum monostearate, magnesium hydroxide, and aluminum hydroxide, zinc salts, tannic acid salts, salts of acids and bases such as sodium and potassium phosphates, sodium and potassium hydroxide, sodium and potassium carbonates and bicarbonates. Preferred surfactants are non-ionic surfactants, such as Cremophor EL, Cremophor RH 40, Cremophor RH 60, polyethylene glycol 1000 succinate, polysorbate 20, polysorbate 80, Solutol HS 15, sorbitan monooleate, poloxamers such as polaxamer 188, Labrafil M-1944CS, Labrafil M-2125CS, Labrasol, Gellucire 44/14, and Softigen 767. Examples of preferred phospholipids are hydrogenated soy phosphatidylcholine, distearoylphosphatidylglycerol, 1-α-dimyristoylphosphatidylcholine, and 1-α-dimyristoylphosphatidylglycerol. Examples of viscosity increasing agents include soluble and insoluble solids such as microcrystalline cellulose and sugar esters.

Should it be necessary to surgically excise the embolism from a site of administration it would be desirable to also include a contrast enhancement agent such as a dye or radiopacity agent in order to allow a healthcare professional to identify the embolus. In addition a contrast agent would assist manufacturing personal in identifying the various strengths of the dosage in preventing manufacturing errors or for cosmetic reasons. Such contrast agents such as dyes, colorants and radiopacity agents are disclosed in the prior art.

Gruber, U.S. Pat. No. 7,214,385 lists dyes added to dosage form useful in identifying abusers by staining the tissues of the abuser. In this invention a dye is considered an aversive agent capable of deterring using the drug for illicit use. In the current invention, the embolizing agent will function to deter the illicit use with or without the contrast agent or dye being present. Therefore, the contrast agent or dye is not anticipated to deter any misuse but rather to assist in identifying an embolism which may or may not be visible to the abuser such as in the case of subcutaneous or intramuscular injection. In addition, it is anticipated that the dye added to the present invention may release from the dosage form whether or not the dosage form is tampered with such as in the case of normal oral use. In a preferred embodiment, the dyes are FD&C dyes. Exemplary FD&C dyes are FD&C Red No. 3, FD&C Red No. 20, FD&C Yellow No. 6, FD&C Blue No. 1, FD&C Blue No. 2, FD&C Green No. 1, FD&C Green No. 3, FD&C Green No. 5, FD&C Red No. 30, D&C Orange No. 5, D&C Red No. 8, D&C Red No. 33, and mixtures thereof.

Preferred embodiments of the invention can include a contrast-enhancing agent that is radiopaque in nature, in particular, a radiopaque material, which exhibits permanent radiopacity, as many metals or metal oxides do. Permanent radiopacity is unlike some other contrast-enhancing agents or radiopaque materials used in embolization or similar medical applications which biodegrade or otherwise lose their effectiveness (radiopacity) over a certain period, e.g., days or weeks, such as 7 to 14 days. (See, e.g., PCT/GB98/02621). Advantage is that permanent radiopaque materials can be monitored or tracked for as long as they remain in the body, whereas other non-permanent contrast-enhancing agents or radiopaque materials have a limited time during which they may be detected and tracked. The preferred concentrations of contrast enhancing agents or dyes are in the range of 0.1%-10% wt/wt of the total composition.

The invention is further described in the following non-limiting examples. In the examples, the invention is illustrated utilizing the drug oxycodone. One skilled in the art will understand that oxycodone is a representative of the drug of the invention and that any drug, especially a drug that may cause an effect unintended by the manufacturer when tampered with, is suitable for the invention.

The invention is further illustrated in the following non-limiting examples.

### Example 1

### Various Oral Gels Containing Embolizing Agents (Amino Methacrylate Copolymer, Methacrylic Acid Copolymer, Type C, Methacrylic Acid Copolymer, Type B, or Methacrylic Acid Copolymer, Type A) were Prepared as Follows

A weighed quantity of the embolizing agent was dissolved in a weighed quantity of plasticizer/plasticizer blend by heating them together to about 70-75° C. with mixing to prepare the embolizing agent gel (blank gel). Weighed quantity of Oxycodone HCl was then added to the blank gel at room temperature and then subsequent drug-loaded gel was heated to 50-60° C. for 10 min. The final formulation was loaded into a two piece gelatin capsule. The compositions of the gels are shown in Table 1.

TABLE 1

| | | | Embolizing |
|---|---|---|---|
| Drug | | | agent:Additives |
| Drug Loading % | Embolizing Agent | Additives | (blank gel) |
| Oxycodone | Amino Methacrylate Copolymer | Triethyl Citrate (TEC) | 40:60 |
| HCl = 20% | | | |
| Oxycodone | Amino Methacrylate Copolymer | Acetyl Triethyl Citrate | 40:60 |
| HCl = 20% | | (ATEC) | |
| Oxycodone | Amino Methacrylate Copolymer | ATEC | 40:60 |
| HCl = 10% | | | |
| Oxycodone | Amino Methacrylate Copolymer: | ATEC | 40:60 |
| Base = 10% | | | |
| Oxycodone | Methacrylic Acid Copolymer, Type A: | ATEC:Tetraglycol | 1:3:3 |
| HCl = 10% | | | |
| Oxycodone | Methacrylic Acid Copolymer, Type C | ATEC:Tetraglycol | 1:3:3 |
| HCl = 10% | | | |
| Oxycodone | Methacrylic Acid Copolymer, Type B | ATEC:Tetraglycol | 1:3:3 |
| HCl = 10% | | | |
| Oxycodone | Amino Methacrylate Copolymer | Benzyl benzoate | 40:60 |
| HCl = 10% | | | |
| Oxycodone | Amino Methacrylate Copolymer | Benzyl benzoate | 60:40 |
| HCl = 10% | | | |
| Oxycodone | Amino Methacrylate Copolymer | Benzyl benzoate | 50:50 |
| HCl = 10% | | | |
| Oxycodone | Amino Methacrylate Copolymer | Benzyl benzoate: | 50:40:10 |
| HCl = 10% | | Isopropyl myristate | |
| Oxycodone | Amino Methacrylate Copolymer | Benzyl benzoate: | 50:40:10 |
| HCl = 10% | | Sisterna SP-01C | |
| Oxycodone | Amino Methacrylate Copolymer | ATBC | 50:50 |
| HCl = 10% | | | |
| Oxycodone | Amino Methacrylate Copolymer | Labrafac Lipophile | 50:50 |
| HCl = 10% | | WL1349 | |

### Example 2

### Effect of the Hydrophilicity/Hydrophobicity of the Plasticizers on the Release of Oxycodone HCl from the Embolizing Agent Containing Gel Prepared with an Embolizing Agent (Amino Methacrylate Copolymer) in SGF (Simulated Gastric Fluid) or Water

Dissolution from the gel formulations listed in Table 2 was tested. The dissolution condition was as follows: UPS method 2, 50 rpm, 900 ml of SGF or water, 37° C. Embolizing agent containing drug loaded gel capsules were put into sinkers. Drug release characteristics from these formulations are shown in FIG. 1 and FIG. 2.

TABLE 2

| | | Drug | Drug per |
|---|---|---|---|
| Drug | Blank gel | loading (%) | capsule (mg) |
| Oxycodone | Amino Methacrylate | 20% | 30 |
| HCl | Copolymer:TEC = 40:60 | | |
| Oxycodone | Amino Methacrylate | 20% | 30 |
| HCl | Copolymer:ATEC = 40:60 | | |

FIGS. 1 and 2 show that drug release from the gel containing ATEC was slower than the release from the gel containing TEC in either SGF or water. The release of drug from both formulations in SGF was much faster than the release in water.

### Example 3

### Effect of Drug Loading on the Release of Oxycodone HCl from the Gel Prepared with an Embolizing Agent (Amino Methacrylate Copolymer) and Plasticizers in SGF or Water

Dissolution from the gel formulations listed in Table 3 was tested. The dissolution condition was as follows: UPS method 2, 50 rpm, 900 ml of SGF or water, 37° C. Gel-loaded capsules were put into sinkers. Drug release characteristics from these formulations are shown in FIG. 3 and FIG. 4.

TABLE 3

| | | Drug | Drug per |
|---|---|---|---|
| Drug | Blank gel | loading (%) | capsule (mg) |
| Oxycodone | Amino Methacrylate | 10% | 30 |
| HCl | Copolymer:ATEC = 40:60 | | |
| Oxycodone | Amino Methacrylate | 20% | 30 |
| HCl | Copolymer:ATEC = 40:60 | | |

As seen in FIG. 3, drug loading level had no significant effect on the release in SGF. FIG. 4 shows that, after 0.5 hour, release from a gel containing 20% drug in water was faster than the release from a gel containing 10% drug.

### Example 4

### Release of Oxycodone HCl from a Gel Prepared with an Embolizing Agent (Amino Methacrylate Copolymer) and ATEC in SGF, pH4.5 PBS, SIF (Simulated Intestinal Fluid) or Water

Dissolution from the gel formulations listed in Table 4 was tested. The dissolution condition was as follows: UPS method 2, 50 rpm, 900 ml of SGF, pH 4.5 PBS, SIF or water, 37° C. Drug loaded gel capsules were put into sinkers. Drug release characteristics from these formulations are shown in FIG. 5.

TABLE 4

| | | Drug | Drug per |
|---|---|---|---|
| Drug | Blank gel | loading (%) | capsule (mg) |
| Oxycodone | Amino Methacrylate | 10% | 30 |
| HCl | Copolymer:ATEC = 40:60 | | |

As seen in FIG. 5, the release of drug from gel in SGF was fastest, followed by the release in pH 4.5 PBS, SIF and water.

### Example 5

### Release of Oxycodone HCl from the Tampered Gel Prepared with an Embolizing Agent (Amino Methacrylate Copolymer) and ATEC in SGF, pH4.5 PBS, SIF or Water

The gel formulation listed in Table 4 was frozen at −20° C. for two hours. The subsequent gel was tampered by grinding with pestle. The drug release from the tampered gel was tested. The dissolution condition was as follows: UPS method 2, 50 rpm, 900 ml of SGF, pH4.5 PBS, SIF or water, 37° C. Drug loaded gel capsules were put into sinkers. Drug release characteristics from these formulations are shown in FIG. 6.

As seen in FIG. 5 and Figure, tampering gels after freezing them did not significantly affect the release in various medium.

### Example 6

### Release of Oxycodone Base from the Gel Prepared with an Embolizing Agent (Amino Methacrylate Copolymer) and ATEC in SGF, pH4.5 PBS, SIF or Water

The dissolution of the formulations listed in Table 5 was tested. The dissolution condition was as follows: UPS method 2, 50 rpm, 900 ml of SGF, pH4.5 PBS, SIF or water, 37° C. Gel-loaded capsules were put into sinkers. Drug release characteristics from these formulations are shown in FIG. 7.

TABLE 5

| | | Drug | Drug per |
|---|---|---|---|
| Drug | Blank gel | loading (%) | capsule (mg) |
| Oxycodone | Amino Methacrylate | 10% | 30 |
| base | Copolymer:ATEC = 40:60 | | |

As seen in FIG. 7, the release of oxycodone base from gel in SGF was fastest, followed, in order, by the release in pH 4.5 PBS, SIF and water.

### Example 7

### Comparison of Drug Release from Different Formulations Before or after Tampering in SGF, pH4.5 PBS, SIF or Water

The dissolution of oxycodone HCl from a commercially available sustained release tablet of oxycodone HCl (Roxicodone®) was tested before and after the tablets were ground. The dissolution condition was as follows: UPS method 2, 50 rpm, 900 ml of SGF, pH4.5 PBS, SIF or water, 37° C. Tablets or ground powder-loaded capsules were put into sinkers. The results were compared to the results of Examples 4 to 6 and were plotted as shown in FIGS. 8, 9, 10, and 11.

As seen in FIGS. 8 to 11, release from crushed Roxicodone tablets was dramatically increased in various dissolution medium compared to the release from intact tablets. The release from oxycodone HCl-loaded embolizing agent containing gel was not significantly affected by crushing. It can also be seen that the release from oxycodone base-loaded gel in water was significantly slower than the release from oxycodone HCl-loaded gel.

### Example 8

### Release of Oxycodone HCl from the Gel Prepared with Embolizing Agent (Methacrylic Acid Copolymer, Type A), ATEC and Tetraglycol in SGF, pH4.5 PBS, SIF or Water

The dissolution of the formulations listed in Table 6 was tested. The dissolution condition was as follows: UPS method 2, 50 rpm, 900 ml of SGF, pH4.5 PBS, SIF or water, 37° C. Gel-loaded capsules were put into sinkers. Drug release characteristics from these formulations are shown in FIG. 12.

TABLE 6

| | | Drug | Drug per |
|---|---|---|---|
| Drug | Blank gel | loading (%) | capsule (mg) |
| Oxycodone | Methacrylic Acid Copolymer, | 10% | 30 |
| HCl | Type A:ATEC:Tetraglycol = | | |
| | 1:3:3 | | |

As seen in FIG. 12, the release from gel prepared with Methacrylic Acid Copolymer, Type A in SIF was fastest, followed in order by the release in SGF, pH 4.5 PBS and water.

### Example 9

### Release of Oxycodone HCl from the Gel Prepared with an Embolizing Agent (Methacrylic Acid Copolymer, Type C), ATEC and Tetraglycol in SGF, pH 4.5 PBS, SIF or Water

The dissolution of the formulations listed in Table 7 was tested. The dissolution condition was as follows: UPS method 2, 50 rpm, 900 ml of SGF, pH4.5 PBS, SIF or water, 37° C. Gel-loaded capsules were put into sinkers. Drug release characteristics from these formulations are shown in FIG. 13.

TABLE 7

| | | Drug | Drug per |
|---|---|---|---|
| Drug | Blank gel | loading (%) | capsule (mg) |
| Oxycodone | Methacrylic Acid Copolymer, | 10% | 30 |
| HCl | Type C:ATEC:Tetraglycol = | | |
| | 1:3:3 | | |

As seen in FIG. 13, the release from gel prepared with Methacrylic Acid Copolymer, Type C in SIF was fastest, follow in order by the release in SGF, pH 4.5 PBS and water.

### Example 10

### Release of Oxycodone HCl from the Gel Prepared with an Embolizing Agent (Methacrylic Acid Copolymer, Type C), ATEC and Tetraglycol in SGF, pH 4.5 PBS, SIF or Water

The dissolution of the formulations listed in Table 8 was tested. The dissolution condition was as follows: UPS method 2, 50 rpm, 900 ml of SGF, pH4.5 PBS, SIF or water, 37° C. Gel-loaded capsules were put into sinkers. Drug release characteristics from these formulations are shown in FIG. 14.

TABLE 8

| | | Drug | Drug per |
|---|---|---|---|
| Drug | Blank gel | loading (%) | capsule (mg) |
| Oxycodone | Methacrylic Acid Copolymer, | 10% | 30 |
| HCl | Type C:ATEC:Tetraglycol = | | |
| | 1:3:3 | | |

As seen in FIG. 14, the release from gel prepared with Methacrylic Acid Copolymer, Type C in SGF or pH14.5 PBS was faster than the release in SIF or water.

### Example 11

### Release of Oxycodone HCl from Roxicodone Tablets or the Gel Prepared with Embolizing Agent (Amino Methacrylate Copolymer) and ATEC in 40% Ethanol

The dissolution of oxycodone HCl from Roxicodone tablets or the gel prepared in Example 5 was tested in 40% ethanol. The dissolution condition was as follows: UPS method 2, 50 rpm, 900 ml of 40% ethanol, 37° C. Tablets or gel-loaded capsules were put into sinkers. Drug release characteristics from these formulations are shown in FIG. 15. As seen in FIG. 15, the release from gel prepared with Amino Methacrylate Copolymer and ATEC in 40% ethanol was much slower than the release from intact Roxicodone tablets.

### Example 12

### Effect of Concentration of an Embolizing Agent (Amino Methacrylate Copolymer) on the Release of Oxycodone HCl from the Gel Prepared with Embolizing Agent (Amino Methacrylate Copolymer) and Plasticizers in SGF or Water

The dissolution from the gel formulations listed in Table 9 was tested. The dissolution condition was as follows: USP method 2, 50 rpm, 900 ml of SGF or water, 37° C.

Gel-loaded capsules were put into sinkers (drug per capsule: 20 mg). Drug release characteristics from these formulations are shown in FIG. 16 and FIG. 17.

TABLE 9

| Drug | Blank gel | Drug (%) |
|---|---|---|
| Oxycodone HCl | Amino Methacrylate Copolymer:Benzyl | 10% |
| | benzoate = 40:60 | |
| Oxycodone HCl | Amino Methacrylate Copolymer:Benzyl | 10% |
| | benzoate = 60:40 | |
| Oxycodone HCl | Amino Methacrylate Copolymer:Benzyl | 10% |
| | benzoate = 50:50 | |

As seen in FIGS. 16 and 17, total drug release in purified water decreased as the amount of embolizing agent was increased. In SGF, total drug release was obtained within 15 minutes in all three concentrations studied, (thicker gel consistency with 60% Amino Methacrylate Copolymer slightly delayed SGF penetration and release was slightly delayed from 10 to minutes to 15 minutes).

### Example 13

### Use of Solvent Blends and Hydrophobic Additives

The dissolution from the gel formulations listed in Table 10 was tested. The dissolution condition was as follows: USP method 2, 50 rpm, 900 ml of SGF or water, 37° C. Drug loaded embolizing agent containing gel (blank gel) capsules were put into sinkers (drug per capsule: 20 mg). Drug release characteristics from these formulations are shown in FIG. 18 and FIG. 19.

TABLE 10

| Drug | Blank gel | Drug (%) |
|---|---|---|
| Oxycodone HCl | Amino Methacrylate Copolymer:Benzyl | 10% |
| | benzoate = 50:50 | |
| Oxycodone HCl | Amino Methacrylate Copolymer:Benzyl | 10% |
| | benzoate:Isopropyl myristate = 50:40:10 | |
| Oxycodone HCl | Amino Methacrylate Copolymer:Benzyl | 10% |
| | benzoate:Sisterna SP-01C = 50:40:10 | |

As seen in FIGS. 18 and 19, addition of hydrophobic sucrose ester Sisterna SP-01C with HLB 1 retarded drug release in SGF and in purified water to a great extent. Replacing benzyl benzoate by isopropyl myristate did not alter drug release in SGF, however, dissolution in water was increased slightly compared to benzyl benzoate alone, (Amino Methacrylate Copolymer is soluble in SGF and formulation dissipates and drug dissolves in either case; but in water, Amino Methacrylate Copolymer being insoluble, gel matrix does retain the integrity and it allows us to see the effect of change in hydrophobicity of solvent blend more clearly).

### Example 14

### Effect of Changing Hydrophobicity of the Plasticizers on the Release of Oxycodone HCl from the Gel Prepared with Embolizing Agent (Amino Methacrylate Copolymer) and Plasticizers in SGF or Water

The dissolution from the gel formulations listed in Table 11 was tested. The dissolution condition was as follows: USP method 2, 50 rpm, 900 ml of SGF or water, 37° C.

Gel-loaded capsules were put into sinkers (drug per capsule: 20 mg). Drug release characteristics from these formulations are shown in FIG. 20 and FIG. 21.

TABLE 11

| Drug | Blank gel | Drug (%) |
|---|---|---|
| Oxycodone HCl | Amino Methacrylate | 10% |
| | Copolymer:Benzyl benzoate = 50:50 | |
| Oxycodone HCl | Amino Methacrylate | 10% |
| | Copolymer:ATBC = 50:50 | |
| Oxycodone HCl | Amino Methacrylate Copolymer:Labrafac | 10% |
| | Lipophile WL1349 = 50:50 | |

As seen in FIGS. 20 and 21, increasing the hydrophobicity of plasticizer decreased dissolution in purified water to great extent.

### Example 15

A powder blend containing oxycodone is prepared as shown in Table 12.

TABLE 12

| Ingredients | Amt/Unit (mg) |
|---|---|
| Oxycodone HCl | 20.0 |
| Spray Dried Lactose | 59.25 |
| Povidone | 5.0 |
| Eudragit RS30D (solids) | 10.0 |
| Triacetin | 2.0 |
| Stearyl Alcohol | 25.0 |
| Talc | 2.5 |
| Magnesium Stearate | 1.25 |
| Opadry Pink Y-S-14518A | 5.0 |

### Process

1. Dispersion: Disperse Eudragit and Triacetin in an aqueous medium to form a Eudragit/Triacetin dispersion.
2. Granulation: Spray the Eudragit/Triacetin dispersion onto the Oxycodone HCl, Spray Dried Lactose and Povidone using a fluid bed granulator.
3. Milling: Discharge the granulation and pass through a mill.
4. Waxing: Melt the stearyl alcohol and add to the milled granulation using a mixer. Allow to cool.
5. Milling: Pass the cooled granulation through a mill.
6. Lubrication: Lubricate the granulation with talc and magnesium stearate using a mixer.

### Example 16

A 20 mg sustained release Oxycodone Formulation is Prepared Containing Thrombin as the Embolizing Agent

### Process

1. Embolizing Agent Addition: Add the stabilized thrombin USP (500 I.U.) to the powder blend from Example 15 and blend using a mixer.
2. Compression: Compress the granulation into tablets using a tablet press.

### Example 17

A substantially non-releasable form of an embolizing agent (thrombin) is prepared by coating thrombin particles with a coating that renders the thrombin substantially non-releasable. The formula of Example 17 is listed in Table 13 below.

TABLE 13

| Ingredients | Amt/Unit (mg) |
|---|---|
| Thrombin | 500 I.U |
| Sugar Spheres (30/35 mesh) | 50.0 |
| Opadry White Y-5-7068 | 2.5 |
| Purified Water | 42.5* |
| OVERCOATING | |
| Opadry White Y-5-7068 | 3.02 |
| Purified Water | 17.11* |
| NON-RELEASE COATING (FOR RENDERING | |
| EMBOLIZING AGENT SUBSTANTIALLY NON- | |
| RELEASABLE) | |
| Eudragit RS30D (dry wt.) | 12.10 |
| Triethyl Citrate | 2.42 |
| Talc | 4.84 |
| Purified Water | 49.21* |
| OVERCOATING | |
| Opadry White Y-5-7068 | 4.12 |
| Purified Water | 23.35* |

*Remains in product as residual moisture only.

### Example 18

A direct compression formulation, as shown in Table 14, for an immediate release opioid analgesic, e.g. oxycodone HCl, tablet having 5 mg of oxycodone HCl was formed by weighing each component separately and mixing the oxycodone HCl and the polymer in a V-blender for about 5 to 10 minutes at low shear conditions or in a high shear blender by mixing 2 to 5 minutes. The other formulation excipients were added to the above blend excepting the lubricant and mixed at the same rate for additional 5 to about 10 minutes. Finally, the lubricant, magnesium stearate was added to the formulation and blended at the same rate for an additional 3 to 5 minutes.

TABLE 14

| Component | Weight (mg) |
|---|---|
| Oxycodone HCl | 5 |
| Polyvinyl alcohol | 160 |
| Avicel PH 102 | 333 |
| Starch 21 | 54 |
| Zinc sulfate | 30 |
| Explotab | 15 |
| Cab-O-Sil | 1.5 |
| Magnesium stearate | 1.5 |

### Example 19

An immediate release Oxycodone tablet is prepared as follows. An embolizing agent (Amino Methacrylate Copolymer), 200 mg was dispersed into the immediate release blend of example 18 and compressed into tablets.

### Example 20

A direct compression formulation, as shown in Table 15, containing an embolizing agent (thrombin 500 I.U.) was formed by weighing each component separately and mixing the thrombin and the polymer in a V-blender for about 5 to 10 minutes at low shear conditions or in a high shear blender by mixing 2 to 5 minutes. The other formulation excipients were added to the above blend excepting the lubricant and mixed at the same rate for additional 5 to about 10 minutes. Finally, the lubricant, magnesium stearate was added to the formulation and blended at the same rate for an additional 3 to 5 minutes.

### Example 21

TABLE 15

| Component | Weight (mg) (Activity/I.U) |
|---|---|
| Thrombin | (500 I.U.) |
| Polyvinyl alcohol | 160 |
| Avicel PH 102 | 333 |
| Starch 21 | 54 |
| Zinc sulfate | 30 |
| Explotab | 15 |
| Cab-O-Sil | 1.5 |
| Magnesium stearate | 1.5 |

A tri layer tablet was prepared by weighing half of the formulation of example 20 and compressing the first layer. A middle layer was then compressed using the formulation of Example 15 a final third layer was then compressed using the remaining material from example 20.

### Example 22

A sustained release oxycodone HCl formulation is prepared having the formula in Table 16 below:

TABLE 16

| Component | Weight (mg) |
|---|---|
| Oxycodone HCl | 10 |
| Eudragit RSPO | 76.5 |
| Ethocel | 4.5 |
| Stearic acid | 27.0 |

### Process

1. Blend milled Stearic acid, ethocel, Oxycodone HCl, and Eudragit RSPO using a V-blender.
2. Extrude the mixture using a Powder Feeder, Melt Extruder (equipped with the 6.times.1 mm die head), Conveyor, Lasermike, and Pelletizer. Powder feed rate—4.2 kg/hr; vacuum—.about.980 mBar Conveyor—such that diameter of extrudate is 1 mm Pelletizer-such that pellets are cut to 1 mm in length
3. Screen pellets using #16 mesh and #20 mesh screens. Collect material that passes through the #16 mesh screen and is retained on the #20 mesh screen.
4. Fill size #2 clear gelatin capsules with the pellets. Range: NLT 114 mg and NMT 126 mg.

One or more embolizing agents as described herein can be incorporated into a capsule with the oxycodone pellets, into the oxycodone pellets, or on the oxycodone pellets by one skilled in the art. The one or more embolizing agents may be in releasable, non-releasable, or substantially non-releasable form or a combination thereof. Preferably, when pellets comprising the embolizing agent(s) are incorporated into the capsule separately from the oxycodone containing pellets they are indistinguishable from the oxycodone pellets.

### Example 23

Four oxycodone formulations were prepared as shown in Table 17.

TABLE 17

| | Oxy- | Amino | | | | |
|---|---|---|---|---|---|---|
| Batch | codone | Methacrylate | | | Carbopol | Carbopol |
| No | base | Copolymer | TEC | ATBC | 971P | 974P |
| 1002-2 | 10 | 20 | 30 | 30 | 10 | |
| 1002-3 | 10 | 20 | 30 | 30 | | 10 |
| 1006-1 | 10 | 20 | | 60 | 10 | |
| 1006-2 | 10 | 20 | | 60 | | 10 |

A weighed quantity of the embolizing agent (amino methacrylate copolymer) was dissolved in a weighed quantity of plasticizer/plasticizer blend (either or both of TEC (slightly hydrophilic) and ATBC (hydrophobic)) by heating them together to about 70-85° C. with mixing to prepare the embolizing agent gel (blank gel). Weighed quantity of Oxycodone base was then added to the heated blank gel and then a sustained releasing agent, either Carbopol 971P (low viscosity) or Carbopol 974P (high viscosity), was suspended in the mixture. The drug-loaded gel was then maintained at 55-65° C. for 10 minutes and was loaded into a two piece gelatin capsule. The compositions of the gels in each capsule are shown in Table 17.

Release of the formulations was determined in simulated blood or tap water (pH 6.8-7.0) at room temperature, in pH 4.5 buffer, in simulated gastric fluid (SGF) at pH 1.2, and in boiling water. Release of drug from each of the formulations in simulated blood or tap water at room temperature following crushing of the capsules was negligible, less than 10% after two hours.

Release in SGF is shown in FIG. 22. As shown, release rate at pH 1.2 can be varied based on hydrophilicity/hydrophobicity of the plasticizer utilized and on the viscosity of gelling agent. The higher viscosity gelling agent provided for more rapid release whereas the lower viscosity gelling agent provided more sustained release. Similarly, a more hydrophilic plasticizer provided more immediate release than a more hydrophilic plasticizer. Release in pH 4.5 buffer is shown in FIG. 23. As shown, release from each formulation was similar to that in SGF.

Table 18 shows release of drug from each of the four formulations after 10 minutes in boiling water. As shown in Table 18, the release rate of all of the formulations, regardless of the hydrophilicity/hydrophobicity of the embolizing liquid and regardless of the viscosity of the sustained releasing agent, was greatly reduced.

TABLE 18

| Batch No | 1002-2 | 1002-3 | 1006-1 | 1006-2 |
|---|---|---|---|---|
| Extraction (%) | 15.6 | 20.7 | 21.8 | 12.8 |

Further modifications, uses, and applications of the invention described herein will be apparent to those skilled in the art. It is intended that such modifications be encompassed in the following claims.

1. A dosage form for oral administration comprising a therapeutically effective amount of a drug that is susceptible to abuse and an effective amount of an embolizing agent wherein, following tampering of the dosage form and administration of the tampered dosage form to a non-oral site of administration, the embolizing agent forms an embolus or a coagulation at the site of the administration.

2. The dosage form of claim 1 which is a tablet.

3. The dosage form of claim 1 which is a capsule.

4. The dosage form of claim 1 wherein the drug is an analgesic.

5. The dosage form of claim 4 wherein the analgesic is an opioid analgesic.

6. The dosage form of claim 5 wherein the opioid analgesic is selected from the group consisting of morphine, codeine, buprenorphine, hydrocodone, oxymorphone, hydromorphone, tramadol, and oxycodone, or a pharmaceutically acceptable salt.

7. The dosage form of claim 1 wherein the drug is selected from the group consisting of a tranquilizer, a CNS depressant, a CNS stimulant, a sedative hypnotic, and a respiratory agent.

8. The dosage form of claim 1 wherein the embolizing agent is pH sensitive.

9. The dosage form of claim 8 wherein the pH sensitive embolizing agent is a polymer.

10. The dosage form of claim 9 wherein the pH sensitive polymer is selected from the group consisting of copolymers of methyl and butyl methacrylate and dimethylaminoethyl methacrylates, methacrylic acid copolymer dispersion, methacrylic acid copolymer, Type B, methacrylic acid copolymer, Type A, methacrylic acid copolymer, Type C, cellulose acetate trimellitate, polyvinyl acetate phthalate, hydroxypropyl methylcellulose phthalate, methacrylic acid/ethyl acrylate copolymers, hydroxypropyl methylcellulose succinate-L, hydroxypropyl methylcellulose succinate-M, hydroxypropyl methylcellulose succinate-H, and cellulose acetate phthalate.

11. The dosage form of claim 1 wherein the embolizing agent is temperature sensitive.

12. The dosage form of claim 11 wherein the temperature sensitive embolizing agent is a polymer.

13. The dosage form of claim 1 wherein the embolizing agent is an agent that causes formation of an embolus when contacted with a biological fluid other than gastric fluid.

14. The dosage form of claim 13 wherein the embolizing agent is thrombin or fibrinogen.

15. The dosage form of claim 1 wherein the embolizing agent is sequestered.

16. The dosage form of claim 13 wherein the sequestered embolizing agent is in the form of a multiplicity of individually coated particles, wherein the coating prevents release of the sequestered embolizing agent when the dosage form has not been tampered.

17. The dosage form of claim 16 wherein the sequestered embolizing agent is dispersed in a matrix that contains a sequestering material that inhibits release of the embolizing agent when the dosage form has not been tampered.

18. The dosage form of claim 1 wherein the presence of the embolizing agent in the dosage form does not decrease the release of the drug from the dosage form when the dosage form is not tampered with and when the dosage form is orally administered.

19. A method for inhibiting abuse of an oral dosage form containing a drug subject to abuse comprising providing a dosage form for oral administration which dosage form comprises a therapeutically effective amount of a drug that is susceptible to abuse and an effective amount of an embolizing agent wherein, following tampering of the dosage form and administration of the tampered dosage form to a non-oral site of administration, the embolizing agent forms an embolus or a coagulation at the site of the administration.

20. A method for making an oral dosage form that is resistant to abuse comprising combining a therapeutically effective amount of a drug that is susceptible to abuse and an amount of an embolizing agent that is effective to cause the formation of an embolus or a coagulation when the dosage form is tampered with and administered to a non-oral site of administration.
